SEQUENCE LISTING (11 sequences)

SEQ ID NO: 1 - DNA, 1581 bp, Homo sapiens
atggggcacctctcagccccacttcacagagtgcgtgtaccctggcaggggcttctgctcacagcctcacttctaaccttctggaacccgcccaccactgcccagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgagctaagtccagtagtagcaaagccccaaatcaaagccagcaagaccacagtcacaggagataaggactctgtgaacctgacctgctccacaaatgacactggaatctccatccgttggttcttcaaaaaccagagtctcccgtcctcggagaggatgaagctgtcccagggcaacaccaccctcagcataaaccctgtcaagagggaggatgctgggacgtattggtgtgaggtcttcaacccaatcagtaagaaccaaagcgaccccatcatgctgaacgtaaactataatgctctaccacaagaaaatggcctctcacctggggccattgctggcattgtgattggagtagtggccctggttgctctgatagcagtagccctggcatgttttctgcatttcgggaagaccggcagggcaagcgaccagcgtgatctcacagagcacaaaccctcagtctccaaccacactcaggaccactccaatgacccacctaacaagatgaatgaagttacttattctaccctgaactttgaagcccagcaacccacacaaccaacttcagcctccccatccctaacagccacagaaataatttattcagaagtaaaaaagcagtaa

SEQ ID NO: 2 - protein, 526 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 3 - DNA, 1395 bp, Homo sapiens
atggggcacctctcagccccacttcacagagtgcgtgtaccctggcaggggcttctgctcacagcctcacttctaaccttctggaacccgcccaccactgcccagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgagctaagtccagtagtagcaaagccccaaatcaaagccagcaagaccacagtcacaggagataaggactctgtgaacctgacctgctccacaaatgacactggaatctccatccgttggttcttcaaaaaccagagtctcccgtcctcggagaggatgaagctgtcccagggcaacaccaccctcagcataaaccctgtcaagagggaggatgctgggacgtattggtgtgaggtcttcaacccaatcagtaagaaccaaagcgaccccatcatgctgaacgtaaactataatgctctaccacaagaaaatggcctctcacctggggccattgctggcattgtgattggagtagtggccctggttgctctgatagcagtagccctggcatgttttctgcatttcgggaagaccggcagctcaggaccactccaatga

SEQ ID NO: 4 - protein, 464 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGSSGPLQ

SEQ ID NO: 5 - DNA, 1293 bp, Homo sapiens
atggggcacctctcagccccacttcacagagtgcgtgtaccctggcaggggcttctgctcacagcctcacttctaaccttctggaacccgcccaccactgcccagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgataatgctctaccacaagaaaatggcctctcacctggggccattgctggcattgtgattggagtagtggccctggttgctctgatagcagtagccctggcatgttttctgcatttcgggaagaccggcagggcaagcgaccagcgtgatctcacagagcacaaaccctcagtctccaaccacactcaggaccactccaatgacccacctaacaagatgaatgaagttacttattctaccctgaactttgaagcccagcaacccacacaaccaacttcagcctccccatccctaacagccacagaaataatttattcagaagtaaaaaagcagtaa

SEQ ID NO: 6 - protein, 430 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTDNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 7 - DNA, 1107 bp, Homo sapiens
atggggcacctctcagccccacttcacagagtgcgtgtaccctggcaggggcttctgctcacagcctcacttctaaccttctggaacccgcccaccactgcccagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgataatgctctaccacaagaaaatggcctctcacctggggccattgctggcattgtgattggagtagtggccctggttgctctgatagcagtagccctggcatgttttctgcatttcgggaagaccggcagctcaggaccactccaatga

SEQ ID NO: 8 - protein, 368 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTDNALPQENGLSPGAIAGIVIGVVALVALIAVALACFLHFGKTGSSGPLQ

SEQ ID NO: 9 - DNA, 24 bp, Artificial Sequence
  Forward primer of CEACAM1
gcaacaggaccacagtcaagacga

SEQ ID NO: 10 - DNA, 22 bp, Artificial Sequence
  Reverse primer of CEACAM1-3L and -4L
gtggttggagactgagggtttg

SEQ ID NO: 11 - DNA, 21 bp, Artificial Sequence
  Reverse primer of CEACAM1-3S and -4S
tggagtggtcctgagctgccg

(19) United States
(12) Patent Application Publication
(10) Pub. No.: US 2024/0115614 A1
(43) Pub. Date: Apr. 11, 2024

(54) STEM CELL IN WHICH IMMUNE CELL TOLERANCE MODULATOR IS OVEREXPRESSED, AND USE THEREOF

(71) Applicants: THE ASAN FOUNDATION, Seoul (KR); UNIVERSITY OF ULSAN FOUNDATION FOR INDUSTRY COOPERATION, Ulsan (KR)

(72) Inventors: Hun Sik KIM, Seoul (KR); Seong Who KIM, Seoul (KR); Eunbi YI, Seoul (KR); Hyojeong KIM, Seoul (KR); Jung Min KIM, Anseong-si (KR); Woo Seon CHOI, Hanam-si (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 18/262,341

(22) PCT Filed: Jan. 14, 2022
(86) PCT No.: PCT/KR2022/000761

§ 371 Date: Jul. 20, 2023

(30) Foreign Application Priority Data
Jan. 20, 2021 (KR) 10-2021-0008386
Jan. 5, 2022 (KR) 10-2022-0001265

(51) Int. Cl. A61K 35/28; A61P 37/06; C07K 14/705; C12N 5/0775; C12N 15/86

(52) U.S. Cl. CPC A61K 35/28; A61P 37/06; C07K 14/70503; C12N 5/0662; C12N 15/86; C12N 2740/15043

(57) ABSTRACT

A method for treating a cell damage-related disease, including administering a composition to a subject in need thereof. The composition contains, as an active ingredient, one or more selected from the group consisting of stem cells genetically engineered to overexpress a carcinoembryonic antigen-related cell adhesion molecule (CEACAM) family protein, cells differentiated from the stem cells, and components derived from the stem cells.

### TECHNICAL FIELD

The present invention relates to a stem cell, in which an immune cell tolerance modulator is overexpressed, for treating chronic refractory diseases such as graft-versus-host disease, atopic dermatitis, and fibrosis, and, more specifically, to: a stem cell genetically engineered to overexpress a carcinoembryonic antigen-related cell adhesion molecule (CEACAM) family protein to obtain the ability to evade immune responses: a pharmaceutical composition comprising the stem cell as an active ingredient; a method for preparing the stem cell; and the like.

The present application claims priority to and the benefit of Korean Patent Application Nos. 10-2021-0008386 and 10-2022-0001265 filed in the Korean Intellectual Property Office on Jan. 20, 2021 and Jan. 5, 2022, respectively, and all the contents disclosed in the specification and drawings of these applications are incorporated in the present application.

### BACKGROUND ART

Mesenchymal stem cells (MSCs) are excellent in regeneration of damaged tissues and immunoregulatory function, and thus are used as a therapeutic agent not only for spinal cord injury, a heart disease, and a degenerative brain disease, but also a chronic refractory disease such as graft-versus-host disease (GVHD), atopic dermatitis, and fibrosis. In particular, MSCs are excellent in functional recovery of damaged cells after transplantation and immunoregulatory function, and thus are used as an important source of therapeutic agents for various refractory diseases, but have limitations in use as a stem cell therapeutic agent because the survival rate of transplanted MSCs sharply decreases due to aging and immunogenic reactions when the MSCs are exposed to an inflammatory environment after in vivo transplantation. Therefore, the survival enhancement technology for MSCs themselves is currently emerging as the most fundamental solution capable of overcoming the limitations of stem cell therapeutic agents.

MSCs are known to die rapidly through interactions with ULBPs, PVR and nectin-2, which are ligands for natural killer cell (NK cell) receptors. Since the survival rate of MSCs is directly associated with therapeutic effect, there is an urgent need for developing a technique for evading immune responses mediated by the natural killer cells in order to enhance the therapeutic effect by improving the survival rate of MSCs themselves. According to these technical needs, genetically engineered MSCs in which receptors, growth factors, cytokines, and the like are overexpressed have recently been developed to enhance immune evasion and a therapeutic effect in MSC transplantation.

Meanwhile, CEACAM1 is a type 1 membrane protein, which has 12 isoforms due to alternative splicing, and is expressed in various immune cells and cancer cells. However, little is known about the functions of CEACAM family proteins in MSCs.

### Disclosure

### Technical Problem

An object of the present invention is to provide a stem cell genetically engineered to overexpress a carcinoembryonic antigen-related cell adhesion molecule (CEACAM) family protein to obtain the ability to evade immune responses.

Another object of the present invention is to provide a pharmaceutical composition for preventing or treating a cell damage-related disease, comprising, as an active ingredient, a stem cell genetically engineered to overexpress a CEACAM family protein or a cell differentiated from the stem cell.

Still another object of the present invention is to provide a method for preparing a stem cell genetically engineered to overexpress a CEACAM family protein to obtain the ability to evade immune responses.

However, the technical objects which the present invention intends to achieve are not limited to the technical objects which have been mentioned above, and other technical objects which have not been mentioned will be clearly understood by a person with ordinary skill in the art to which the present invention pertains from the following description.

### Technical Solution

In order to achieve the object of the present invention as described above, the present invention provides a stem cell genetically engineered to overexpress a carcinoembryonic antigen-related cell adhesion molecule (CEACAM) family protein to obtain the ability to evade immune responses.

Further, the present invention provides a composition comprising a stem cell genetically engineered to overexpress a CEACAM family protein to obtain the ability to evade immune responses.

In an exemplary embodiment of the present invention, the CEACAM family protein may be one or more selected from the group consisting of CEACAM1, CEACAM3, CEACAM5, and CEACAM6, but is not limited thereto.

In another exemplary embodiment of the present invention, the CEACAM family protein may be CEACAM1.

In still another exemplary embodiment of the present invention, the CEACAM1 may be one or more selected from the group consisting of CEACAM1-3L, CEACAM1-3S, CEACAM1-4L and CEACAM1-4S, but is not limited thereto.

In yet another exemplary embodiment of the present invention, the stem cell may be mesenchymal stem cells, but is not limited thereto.

In yet another exemplary embodiment of the present invention, the mesenchymal stem cells may be one or more selected from the group consisting of bone marrow-derived mesenchymal stem cells, adipose-derived mesenchymal stem cells, umbilical cord-derived mesenchymal stem cells, umbilical cord blood-derived mesenchymal stem cells, embryonic stem cell-derived mesenchymal stem cells, and induced pluripotent stem cell-derived mesenchymal stem cells, but are not limited thereto.

In yet another exemplary embodiment of the present invention, the stem cell may further overexpress an immune checkpoint protein.

In yet another exemplary embodiment of the present invention, the immune checkpoint protein may be one or more selected from the group consisting of PD-1, PD-L1, PD-L2, CD47, CD39, CD73, CD200, HVE4. CD155, TIM3, LAG-3, CTLA-4, A2AR, B7-H3, B7-H4, HLA-E, BTLA, IDO, KIR, and VISTA, but is not limited thereto.

In yet another exemplary embodiment of the present invention, the stem cell may evade an immune response by natural killer (NK) cells.

In yet another exemplary embodiment of the present invention, the evasion of the immune response by NK cells may be caused by a decrease in degranulation activity of NK cells, but is not limited thereto.

In yet another exemplary embodiment of the present invention, the stem cell may suppress the proliferation of T cells.

In yet another exemplary embodiment of the present invention, the stem cell may have an improved in vivo survival rate.

In addition, the present invention provides a pharmaceutical composition for preventing or treating a cell damage-related disease, comprising, as an active ingredient, one or more selected from the group consisting of stem cells genetically engineered to overexpress a carcinoembryonic antigen-related cell adhesion molecule (CEACAM) family protein, cells differentiated from the stem cells, and components derived from the stem cells.

Furthermore, the present invention provides a method for preventing or treating a cell damage-related disease, the method comprising administering a composition comprising, as an active ingredient, one or more selected from the group consisting of stem cells genetically engineered to overexpress a carcinoembryonic antigen-related cell adhesion molecule (CEACAM) family protein, cells differentiated from the stem cells, and components derived from the stem cells to a subject in need thereof.

Further, the present invention provides a use of a composition comprising, as an active ingredient, one or more selected from the group consisting of stem cells genetically engineered to overexpress a carcinoembryonic antigen-related cell adhesion molecule (CEACAM) family protein, cells differentiated from the stem cells, and components derived from the stem cells for preventing or treating a cell damage-related disease.

In addition, the present invention provides a use of a composition comprising, as an active ingredient, one or more selected from the group consisting of stem cells genetically engineered to overexpress a carcinoembryonic antigen-related cell adhesion molecule (CEACAM) family protein, cells differentiated from the stem cells, and components derived from the stem cells for preparing a drug for treating a cell damage-related disease.

Furthermore, the present invention provides a cell therapeutic agent comprising, as an active ingredient, stem cells genetically engineered to overexpress a CEACAM family protein or cells differentiated from the stem cells.

Further, the present invention provides a method for preventing or treating a cell damage-related disease, the method comprising administering the cell therapeutic agent to a subject in need thereof.

In addition, the present invention provides a use of the cell therapeutic agent for preventing or treating a cell damage-related disease.

Furthermore, the present invention provides a use of the cell therapeutic agent for preparing a drug for treating a cell damage-related disease.

In an exemplary embodiment of the present invention, the cell damage-related disease may be one or more selected from the group consisting of inflammatory diseases, autoimmune diseases, neurodegenerative diseases, and graft-versus-host disease, but is not limited thereto.

In another exemplary embodiment of the present invention, the inflammatory disease may be any one or more selected from the group consisting of atopic dermatitis, systemic lupus erythematosus, lupus, lupus pernio, lupus tuberculosis, lupus nephritis, dystrophic epidermolysis bullosa, psoriasis, rheumatic fever, rheumatoid arthritis, lumbago, fibromyalgia, myofascial diseases, undifferentiated spondyloarthropathy, undifferentiated arthropathy, arthritis, inflammatory osteolysis, reactive arthritis, osteoarthritis, scleroderma, osteoporosis, chronic inflammatory diseases caused by viral or bacterial infection, colitis, ulcerative colitis, inflammatory bowel disease, fungal infections, burns, wounds caused by surgical or dental surgery, diabetic foot ulcers, type 1 diabetes, type2 diabetes, ulcerative skin diseases, sinusitis, rhinitis, conjunctivitis, asthma, dermatitis, inflammatory collagen vascular disease, glomerulonephritis, encephalitis, inflammatory enteritis, chronic obstructive pulmonary disease, bronchiolitis obliterans, sepsis, septic shock, pulmonary fibrosis, atherosclerosis, myocarditis, endocarditis, pericarditis, cystic fibrosis, Hashimoto's thyroiditis, Graves' disease, leprosy, syphilis, Lyme disease, Borreliosis, neurological-Borreliosis, tuberculosis, sarcoidosis, macular degeneration, uveitis, irritable bowel syndrome, Crohn's disease, Sjogren's syndrome, chronic fatigue syndrome, chronic fatigue immune dysfunction syndrome, myalgic encephalomyelitis, amyotrophic lateral sclerosis, Parkinson's disease, and multiple sclerosis, but is not limited thereto.

In still another exemplary embodiment of the present invention, the autoimmune disease may be any one or more selected from the group consisting of autoimmune hepatitis, rheumatoid arthritis, osteoarthitis, insulin dependent diabetes mellitus, ulcerative colitis, Crohn's disease, multiple sclerosis, autoimmune myocarditis, scleroderma, myasthenia gravis, polymyositis, dermatomyositis, Hashimoto's disease, autoimmune cytopenia, Sjogren's syndrome, vasculitis syndrome, and systemic lupus erythematosus, but is not limited thereto.

In yet another exemplary embodiment of the present invention, the neurodegenerative disease may be any one or more selected from the group consisting of Alzheimer's disease, dementia, multi-infarct dementia, frontotemporal dementia, dementia with Lewy bodies, mild cognitive impairment, corticobasal degeneration, Parkinson's disease, depression, a metabolic brain disease, multiple system atrophy, Huntington's disease, progressive supranuclear palsy, epilepsy, spinal muscular atrophy, dentatorubropallidoluysian atrophy, spinocerebellar ataxia, glaucoma, stroke, cerebral ischemia, postencephalitic parkinsonism, Tourette syndrome, restless leg syndrome, attention-deficit hyperactivity disorder, Kennedy's disease, amyotrophic lateral sclerosis, multiple sclerosis, primary lateral sclerosis, and progressive bulbar palsy, but is not limited thereto.

In yet another exemplary embodiment of the present invention, the CEACAM family protein may be CEACAM1, but is not limited thereto.

Further, the present invention provides a method for preparing a stem cell genetically engineered to overexpress a carcinoembryonic antigen-related cell adhesion molecule (CEACAM) family protein, the method comprising (a) cloning a viral vector expressing a CEACAM family protein: (b) preparing a vius comprising the vector; and (c)overexpressing the CEACAM family protein by infecting stem cells with the virus prepared in Step (b).

In an exemplary embodiment of the present invention, the viral vector may be one or more selected from the group consisting of a lentiviral vector, a retroviral vector, an adenoviral vector, and a paramyxovirus vector, but is not limited thereto.

### Advantageous Effects

A stem cell and a pharmaceutical composition comprising the same as an active ingredient, according to the present invention, have the effects of reducing the degranulation and cell-killing activity of NK cells and increasing the survival rate of stem cells. Therefore, it is expected that if a stem cell in which CEACAM protein is overexpressed and an immune escape function is obtained, according to the present invention, repeated administration-induced side effects are reduced by increasing the in vivo survival rate thereof and, simultaneously, use as a cell therapeutic agent effective for various inflammatory diseases, autoimmune diseases, and the like, such as graft-versus-host disease, asthma, and fibrosis is possible.

### DESCRIPTION OF DRAWINGS

FIGS. 1A and 1B are views for verifying the individual isoform expression of CEACAM1 in umbilical cord-derived mesenchymal stem cells (UC-MSCs), FIG. 1A illustrates flow cytometry results and FIG. 1B illustrates reverse transcription-polymerase chain reaction (RT-PCR) results (4L-MSC, CEACAM1-4L-overexpressing MSC; 4S-MSC, CEACAM1-4S-overexpressing MSC; 3L-MSC, 20 CEACAM1-3L-overexpressing MSC; and 3S-MSC, CEACAM1-3S-overexpressing MSC, hereinafter the same).

FIG. 2 is a view confirming the CEACAM1 expression pattern according to the treatment of NK, NKT, and T cells in peripheral blood mononuclear cells (PBMCs) with cytokines (PHA-P; and IL-12, IL-2, IL-15, or IFN7).

FIGS. 3A and 3B are views confirming the ENK cytotoxic resistance of CEACAM1-overexpressing stem cells, FIG. 3A is a graph comparatively analyzing the survival rate of CEACAM1-overexpressing UC-MSCs, and FIG. 3B shows the results of analyzing the degranulation activity of ENK cells in terms of CD107a expression.

FIGS. 4A and 4B are views confirming the cytotoxic resistance caused by NK cells in peripheral blood mononuclear cells (PBMCs) of CEACAM1-overexpressing stem cells, FIG. 4A is a graph comparatively analyzing the survival rate of CEACAM1-overexpressing UC-MSCs, and FIG. 4B shows the results of analyzing the degranulation activity of NK cells in terms of CD107a expression.

FIG. 5 is a view confirming the cytotoxicity resistance of CEACAM1-overexpressing stem cells by an NK92 cell line.

FIGS. 6A and 6B are views confirming the ability of CEACAM1-overexpressing stem cells to suppress the proliferation of CD4 T cells and CD8 T cells,

FIG. 6A shows the results of analyzing the proliferation rate of CD4 T cells, and FIG. 6B shows the results of analyzing the proliferation rate of CD8 T cells (PBMCs+PHA, PBMCs treated with PHA-P alone, hereinafter the same).

FIGS. 7A to 7F are views additionally confirming the ability of CEACAM1-overexpressing stem cells to suppress the proliferation of CD4 T cells (FIGS. 7A to 7C) and CD8 T cells (FIGS. 7D to 7F), FIGS. 7A and 7D show the results of comparing CFSE fluorescence intensity, FIGS. 7B and 7E show the results of measuring the number and degree of divisions of T cells, and FIGS. 7C and 7F show the results of analyzing CD25 levels.

FIGS. 8A and 8B are views confirming the ability of CEACAM1-overexpressing stem cells to suppress the cytokine production of CD4 T cells and CD8 T cells, FIG. 8A shows the results of analyzing IFNγ and TNFα levels in CD4 T cells, and FIG. 8B shows the results of analyzing IFNγ and TNFα levels in CD8 T cells (EV-MSC indicates empty-MSCs).

FIGS. 9A and 9B show the effects of CEACAM1-overexpressing stem cells in treating graft-versus-host disease using an animal model, FIG. 9A shows the results of analyzing the changes in body weights of graft-versus-host disease mouse models into which CEACAM1-overexpressing stem cells are injected over time, and FIG. 9B shows the result of analyzing the survival rate of the mouse models over time.

### MODES OF THE INVENTION

The present inventors produced genetically engineered stem cells in which CEACAM1 was overexpressed to measure their immune evasion ability against immune cells, particularly, natural killer cells (NK cells), and as a result, they confirmed that the survival rate of CEACAM1-overexpressing stem cells was increased, thereby completing the present invention.

In an exemplary embodiment of the present invention, it was confirmed that various isoforms of CEACAM1 were overexpressed in umbilical cord-derived mesenchymal stem cells (UC-MSCs) using a lentivirus, and each CEACAM1 isoform was specifically expressed as intended in the present invention (see Example 1).

In another exemplary embodiment of the present invention, CEACAM1 expression patterns for each cell were analyzed, and it was confirmed that CEACAM1 expression was increased according to immune cells when cells were treated with a combination of PHA-P and various cytokines, and particularly, that the CEACAM1 expression of T cells was further increased (see Example 2).

In still another exemplary embodiment of the present invention, the cell survival rate was increased in mesenchymal stem cells by CEACAM1 overexpression, and it was proved that CEACAM1 is a key factor in evading the immune responses of ENK cells (see Example 3).

In yet another exemplary embodiment of the present invention, it was confirmed that the cytotoxic ability of NK cells was reduced due to CEACM1 of MSCs, proving that NK cells in PBMCs influence MSC cell death and CEACM1 expression is a key factor in evading MSC cell death (see Examples 4 and 5).

In yet another exemplary embodiment of the present invention, it was confirmed that CEACAM1-overexpressing MSCs have the ability to suppress the proliferation of T cells, and in particular, are more dominant in suppressing CD4 T cell proliferation than CD8 T cell proliferation (see Examples 6 and 7).

In yet another exemplary embodiment of the present invention, it was confirmed that CEACAM1-overexpressing MSCs have the effect of suppressing the cytokine production of CD4 and CD8 T cells, which can inhibit the survival rate of mesenchymal stem cells (see Example 8).

In yet another exemplary embodiment of the present invention, it was confirmed at an in vivo level that CEACAM1-overexpressing MSCs have excellent therapeutic effects against immune diseases such as graft-versus-host disease using a graft-versus-host disease animal model (see Example 9).

Therefore, the present invention may provide a stem cell genetically engineered to overexpress a carcinoembryonic antigen-related cell adhesion molecule (CEACAM) family protein to obtain the ability to evade immune responses.

As another aspect of the present invention, the present invention may provide a stem cell composition genetically engineered to overexpress a CEACAM family protein to obtain the ability to evade immune responses.

As used herein, the term genetic engineering or genetically engineered refers to the act of introducing one or more genetic modifications into a cell, or cells prepared by the same. For example, the stem cells may be genetically engineered to increase the expression or activity of a CEACAM family protein or an active fragment thereof, and may comprise an exogenous gene encoding CEACAM1 or an active fragment thereof.

The increase in activity may mean that the activity of the same type of protein or enzyme is greater than the activity of the endogenous protein or enzyme that a given non-genetically engineered parent cell (for example: wild-type) has or does not have.

The exogenous gene may be expressed in an amount sufficient for the activity of the protein mentioned above to be increased in the mesenchymal stem cell or host cell compared to a parent cell thereof. The exogenous gene may be introduced into a parent cell via an expression vector. Further, the exogenous gene may be introduced in the form of a linear polynucleotide into a mother cell. In addition, the exogenous gene may be expressed in cells from an expression vector (for example: plasmid). Furthermore, the exogenous genes may be inserted into and expressed in a genetic material (for example: chromosome) in cells for stable expression.

The CEACAM family protein or an active fragment thereof may be prepared as a fusion protein. In the method of preparing a fusion protein, a polynucleotide encoding a CEACAM family protein or an active fragment thereof may be ligated in frame with a polynucleotide encoding another protein or peptide, which may be inserted into an expression vector for expression in a host. Techniques known in the art are available for such purposes. For peptides fused with the CEACAM family protein, known peptides such as FLAG, 6× His residues consisting of 6 histidines (His), 10× His, influenza hemagglutinin (HA), a human sync fragment, a VSV-GP fragment, a p18HIV fragment, T7-tag, HSV-tag, E-tag, an SV40T antigen fragment, lck tag, an alpha-tubulin fragment, B-tag, and a Protein C fragment may be used. In addition, a CEACAM family protein or an active fragment thereof can be ligated with glutathione-S-transferase (GST), influenza hemagglutinin (HA), immunoglobulin constant regions, beta-galactosidase, maltose-binding protein (MBP), and the like in order to make a fusion protein.

In the present invention, the CEACAM family protein may be one or more selected from the group consisting of CEACAM1. CEACAM3, CEACAM4, CEACAM5. CEACAM6, CEACAM7, CEACAM8, CEACAM16, CEACAM18, CEACAM19, CEACAM20, and CEACAM21, may be preferably one or more selected from the group consisting of CEACAM1, CEACAM3, CEACAM5, and CEACAM6, and may be more preferably CEACAM1, but is not limited thereto.

As used herein, the term CEACAM1 is used to refer to a protein product of the CEACAM1 gene, for example, NP_001020083.1, NP_001703.2. To date, 12 different CEACAM1 splice variants have been detected in humans. Individual CEACAM1 isoforms differ with respect to the number of extracellular immunoglobulin-like domains (for example, CEACAM1 with four extracellular immunoglobulin-like domains is known as CEACAM1-4), membrane anchorage and/or the length of their cytoplasmic tail (for example, CEACAM1-4 with a long 5 cytoplasmic tail is known as CEACAM1-4L, and CEACAM1-4 with a short cytoplasmic tail is known as CEACAM1-4S). The N-terminal domain of CEACAM1 starts immediately after the signal peptide and its structure is regarded as the IgV-type. For example, in CEACAM1 annotation P13688, the N-terminal IgV-type domain consists of 108 amino acids, from amino acid 35 to 142. This domain was identified as being responsible for homophilic binding activity ([Watt et al., 2001, Blood. 98, 1469-79]). All variants, comprising these splice variants are comprised within the term “CEACAM1.”

In the present invention, the CEACAM1 may be one or more selected from the group consisting of CEACAM1-3L, CEACAM1-3S, CEACAM1-4L and CEACAM1-4S, but is not limited thereto.

Preferably, the CEACAM1-4L protein according to the present invention may comprise an amino acid sequence of SEQ ID NO: 2, preferably consist of the amino acid sequence of SEQ ID NO: 2, but is not limited thereto, and variants of the amino acid sequence are comprised within the scope of the present invention. That is, the polypeptide consisting of SEQ ID NO: 2 of the present invention is an functional equivalent of the polypeptide that constitutes the same, and is a concept comprising variants capable of performing functionally the same action as the polypeptide, for example, even though a partial amino acid sequence of the polypeptide is modified by deletion, substitution or insertion. Specifically, the polypeptide encoding the CEACAM1-4L protein may comprise a nucleotide sequence having a sequence homology of 70% or more, more preferably 80% or more, even more preferably 90% or more, and most preferably 95% or more to any one amino acid sequence represented by SEQ ID NO: 2. For example, the polypeptide comprises a polypeptide having a sequence homology of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. The % sequence homology to a polypeptide is confirmed by comparing a comparison region with an optimally aligned sequence, and a portion of the polypeptide sequence in the comparison region may comprise an addition or deletion (that is, a gap) compared to the reference sequence (does not comprise additions or deletions) for the optimal alignment of the two sequences. Furthermore, the CEACAM1-4L protein according to the present invention may be encoded by a polynucleotide comprising a nucleic acid sequence of SEQ ID NO: 1, and may be preferably encoded by a polynucleotide consisting of a nucleic acid sequence of SEQ ID NO: 1, but is not limited thereto, and variants of the polynucleotide are comprised within the scope of the present invention.

Preferably, the CEACAM1-4S protein according to the present invention may be encoded by a polypeptide comprising an amino acid sequence of SEQ ID NO: 4 or consisting of the amino acid sequence of SEQ ID NO: 4, but is not limited thereto. That is, the CEACAM1-4S protein may comprise an amino acid sequence having a sequence homology of 70% or more, more preferably 80% or more, even more preferably 90% or more, and most preferably 95% or more to the amino acid sequence represented by SEQ ID NO: 4. Further, the CEACAM1-4S protein according to the present invention may be encoded by a polynucleotide comprising a nucleic acid sequence of SEQ ID NO: 3, and may be preferably encoded by a polynucleotide consisting of a nucleic acid sequence of SEQ ID NO: 3, but is not limited thereto, and variants of the polynucleotide are comprised within the scope of the present invention.

Preferably, the CEACAM1-3L protein according to the present invention may be encoded by a polypeptide comprising an amino acid sequence of SEQ ID NO: 6 or consisting of the amino acid sequence of SEQ ID NO: 6, but is not limited thereto. That is, the CEACAM1-3L protein may comprise an amino acid sequence having a sequence homology of 70% or more, more preferably 80% or more, even more preferably 90% or more, and most preferably 95% or more to the amino acid sequence represented by SEQ ID NO: 6. In addition, the CEACAM1-3L protein according to the present invention may be encoded by a polynucleotide comprising a nucleic acid sequence of SEQ ID NO: 5, and may be preferably encoded by a polynucleotide consisting of a nucleic acid sequence of SEQ ID NO: 5, but is not limited thereto, and variants of the polynucleotide are comprised within the scope of the present invention.

Preferably, the CEACAM1-3S protein according to the present invention may be encoded by a polypeptide comprising an amino acid sequence of SEQ ID NO: 8 or consisting of the amino acid sequence of SEQ ID NO: 8, but is not limited thereto. That is, the CEACAM1-3S protein may comprise an amino acid sequence having a sequence homology of 70% or more, more preferably 80% or more, even more preferably 90% or more, and most preferably 95% or more to the amino acid sequence represented by SEQ ID NO: 8. Furthermore, the CEACAM1-3S protein according to the present invention may be encoded by a polynucleotide comprising a nucleic acid sequence of SEQ ID NO: 7, and may be preferably encoded by a polynucleotide consisting of a nucleic acid sequence of SEQ ID NO: 7, but is not limited thereto, and variants of the polynucleotide are comprised within the scope of the present invention.

In the present invention, the stem cell may be mesenchymal stem cells, but is not limited thereto.

As used herein, the term mesenchymal stem cell (MSC) may refer to a cell that maintains self-renewal and stemness and can differentiate into various mesenchymal tissues, and may comprise mesenchymal stem cells of animals comprising mammals, for example, humans.

The mesenchymal stem cells may be isolated from a tissue such as an embryonic sac, the placenta, umbilical cord blood, skin, peripheral blood, bone marrow, fat, muscle, liver, nerve tissue, the periosteum, a fetal membrane, a synovial membrane, synovial fluid, the amnion, semilunar cartilage, an anterior cruciate ligament, articular chondrocytes, deciduous teeth, pericytes, trabecular bone, subpatellar fat mass, the spleen, and the thymus, and preferably, bone marrow-derived mesenchymal stem cells, adipose-derived mesenchymal stem cells, umbilical cord-derived mesenchymal stem cells, umbilical cord blood-derived mesenchymal stem cells, embryonic stem cell-derived mesenchymal stem cells, induced pluripotent stem cell-derived mesenchymal stem cells, and the like may be used, but are not limited thereto, and the origin of stem cells may be appropriately selected by those skilled in the art according to the therapeutic purpose and desired effects.

Further, for the mesenchymal stem cells, cultures or lysates thereof or extracts thereof may be alternatively used. The cultures, lysates, or extracts may be useful alternatives when it is difficult to use the cells as they are, and comprise cellular constituents, comprising proteins and the like, and thus may exhibit a biological activity similar or equivalent to that of native cells. The lysates or extracts may be obtained using commercially available cell lysis kits or extraction kits.

In the present invention, the stem cells may additionally overexpress immune checkpoint proteins, and non-limiting examples of the immune checkpoint proteins comprise PD-1, PD-L1, PD-L2, CD47, CD39, CD73, C1200, HVEM, CD155, TIM3, LAG-3, CTLA-4, A2AR, B7-H3, B7-H4, HLA-E, BTLA, IDO, KIR, VISTA, combinations thereof, and the like. The stem cells according to the present invention may have additional immunoregulatory functions as well as a more enhanced survival rate by additionally expressing the immune checkpoint proteins.

In the present invention, the stem cell may evade an immune response by natural killer (NK) cells.

As used herein, the term natural killer cells or NK cells is defined as a large granular lymphocyte (LGL) as a cytotoxic lymphocyte constituting a major component of the innate immune system, and constitutes a third type of cell, other than B and T lymphocytes, differentiated from a common lymphoid progenitor (CLP). The natural killer cells or NK cells comprises natural killer cells without additional modification derived from any tissue source, and may comprise not only mature natural killer cells but also natural killer progenitor cells. The natural killer cells are activated by a response to interferon or macrophage-derived cytokines, and the natural killer cells comprise two types of surface receptors, which are called activating receptors and inhibitory receptors and control the cytotoxic activity of the cells. Natural killer cells may be produced from any source, for example, hematopoietic cells from placental tissue, placental perfusate, cord blood, placental blood, peripheral blood, the spleen, the liver, and the like, for example, hematopoietic stem or progenitor cells.

As used herein, the term immune response evasion refers to the suppression of the immune system of a subject (or host) or components thereof (for example, response of NK cells) by stem cells to maximize or allow the viability of exogenously administered/transplanted stem cells

In the present invention, the evasion of the immune response by NK cells may be caused by a decrease in degranulation activity of NK cells, but is not limited thereto.

In the present invention, the stem cells may suppress the proliferation of T-cells, and bring about an effect of improving the in vivo survival rate of stem cells together with a reduction in NK cell activity.

As still another aspect of the present invention, the present invention provides a pharmaceutical composition for preventing or treating a cell damage-related disease, comprising, as an active ingredient, one or more selected from the group consisting of stem cells genetically engineered to overexpress a carcinoembryonic antigen-related cell adhesion molecule (CEACAM) family protein, cells differentiated from the stem cells, and components derived from the stem cells.

That is, the composition according to the present invention is a component derived from stem cells overexpressing a CEACAM family protein, and may be comprised without limitation as long as it can exhibit the characteristics or functions of the stem cells. Specifically, in the present invention, the components derived from stem cells are a concept comprising not only CEACAM-overexpressing stem cells themselves, but also cultures, lysates, and extracts thereof. As used herein, the tenn “culture” comprises a culture solution itself in which the CEACAM-overexpressing stem cells according to the present invention are cultured in a suitable liquid medium, a filtrate (filtrate or centrifuged supernatant) from which CEACAM-overexpressing stem cells are removed by filtering or centrifuging the culture solution, a cell lysate obtained by subjecting the culture solution to ultrasonication or treating the culture solution with lysozyme, and the like, and preferably refers to a supernatant after centrifugation, but is not limited thereto. In addition, the culture solution may comprise both a concentrate of the culture solution and a dried product of the culture solution.

More specifically, components derived from stem cells in the present invention comprise carbohydrates, lipids, proteins (comprising peptides), glycoproteins, oligonucleotides, vitamins, other metabolites, and the like produced from the CEACAM-overexpressing stem cells according to the present invention, and also comprise extracellular vesicles. In the present invention, the extracellular vesicle refers to a membrane structure with a size of tens to hundreds of nanometers (preferably about 20 to 300 nm) consisting of a double phospholipid membrane having the same structure as the cell membrane (provided that the particle size of exosomes may vary depending on the stem cell type to be isolated, isolation method and measurement method). The extracellular vesicles comprise various sugars, proteins, miRNAs, mRNAs, and DNAs produced by cells as cargo, and this cargo is specific to each cell type. As such, since the extracellular vesicles are secreted from cells comprising biologically active materials produced by the cells, the extracellular vesicles share the characteristics of the corresponding cells and can therefore exhibit the biological activity of the cells by themselves. In the present invention, the meaning of extracellular vesicles comprises exosomes and microvesicles, membrane vesicles, ectosomes, shedding vesicles, microparticles, or an equivalent thereof.

As yet another aspect of the present invention, the present invention provides a cell therapeutic agent comprising, as an active ingredient, stem cells genetically engineered to overexpress a CEACAM family protein or cells differentiated from the stem cells.

As used herein, the term “cell therapeutic agent” refers to a drug used for the purpose of treatment, diagnosis, and prevention, by using a cell or tissue prepared through isolation from a human, culture and specific manipulation (US FDA regulations), and specifically, it refers to a drug used for the purpose of treatment, diagnosis, and prevention through a series of actions of in vitro multiplying and/or sorting living autologous, allogenic and xenogeneic cells or changing the biological characteristics of cells by other methods for the purpose of recovering the functions of cells or tissues. Cell therapeutic agents are largely classified into somatic cell therapeutic agents and stem cell therapeutic agents according to the degree of cell differentiation, and the present invention particularly relates to a stem cell therapeutic agent.

In the present invention, the cell damage-related disease may be one or more selected from the group consisting of inflammatory diseases, autoimmune diseases, neurodegenerative diseases, and graft-versus-host disease, but is not limited thereto.

In the present invention, non-limiting examples of the inflamatory disease comprise atopic dermatitis, systemic lupus erythematosus, lupus, lupus pernio, lupus tuberculosis, lupus nephritis, dystrophic epidermolysis bullosa, psoriasis, rheumatic fever, rheumatoid arthritis, lumbago, fibromyalgia, myofascial diseases, undifferentiated spondyloarthropathy, undifferentiated arthropathy, arthritis, inflamatory osteolysis, reactive arthritis, osteoarthritis, scleroderma, osteoporosis, chronic inflammatory diseases caused by viral or bacterial infection, colitis, ulcerative colitis, inflammatory bowel disease, fungal infections, burns, wounds caused by surgical or dental surgery, diabetic foot ulcers, type 1 diabetes, type 2 diabetes, ulcerative skin diseases, sinusitis, rhinitis, conjunctivitis, asthma, dermatitis, inflammatory collagen vascular disease, glomerulonephritis, encephalitis, inflammatory enteritis, chronic obstructive pulmonary disease, bronchiolitis obliterans, sepsis, septic shock, pulmonary fibrosis, atherosclerosis, myocarditis, endocarditis, pericarditis, cystic fibrosis. Hashimoto's thyroiditis, Graves' disease, leprosy, syphilis, Lyme disease, neurological Lyme disease, tuberculosis, sarcoidosis, macular degeneration, uveitis, irritable bowel syndrome, Crohn's disease, Sjogren's syndrome, chronic fatigue syndrome, chronic fatigue immune dysfunction syndrome, myalgic encephalomyelitis, amyotrophic lateral sclerosis, Parkinson's disease, multiple sclerosis, and the like.

Non-limiting examples of the autoimmune disease comprise autoimmune hepatitis, rheumatoid arthritis, osteoarthitis, insulin dependent diabetes mellitus, ulcerative colitis, Crohn's disease, multiple sclerosis, autoimmune myocarditis, scleroderma, myasthenia gravis, polymyositis, dermatomyositis, Hashimoto's disease, autoimmune cytopenia, Sjogren's syndrome, vasculitis syndrome, systemic lupus erythematosus, and the like.

Non-limiting examples of the neurodegenerative disease comprise Alzheimer's disease, dementia, multi-infarct dementia, frontotemporal dementia, dementia with Lewy bodies, mild cognitive impairment, corticobasal degeneration, Parkinson's disease, depression, metabolic brain diseases, multiple system atrophy, Huntington's disease, progressive supranuclear palsy, epilepsy, spinal muscular atrophy, dentatorubropallidoluysian atrophy, spinocerebellar ataxia, glaucoma, stroke, cerebral ischemia, postencephalitic parkinsonism, Tourette syndrome, restless leg syndrome, attention-deficit hyperactivity disorder, Kennedy's disease, amyotrophic lateral sclerosis, multiple sclerosis, primary lateral sclerosis, progressive bulbar palsy, and the like.

In the present invention, the CEACAM family protein may be any one of CEACAM1. CEACAM3, CEACAM4, CEACAM5, CEACAM6, CEACAM7, CEACAM8, CEACAM16, CEACAM18, CEACAM19, CEACAM20, and CEACAM21 as described above, and may be preferably CEACAM1, but is not limited thereto.

Meanwhile, the content of mesenchymal stem cells overexpressing the CEACAM protein in the pharmaceutical composition of the present invention can be appropriately adjusted according to the symptoms of the disease, the degree of progression of the symptoms, the condition of the patient, and the like, and may be, for example, 0.0001 to 99.9 wt %, or 0.001 to 50 wt %, but is not limited thereto. The content ratio is a value based on a dry amount from which the solvent is removed.

The composition according to the present invention may comprise the active ingredient alone or may be provided as a pharmaceutical composition comprising one or more pharmaceutically acceptable carriers, excipients or diluents.

Specifically, the carrier may be, for example, a colloidal suspension, a powder, a saline solution, a lipid, a liposome, a microsphere or a nonspherical particle. These may be complexed or associated with delivery vehicles, and may be transported using delivery systems known in the art, such as lipids, liposomes, microparticles, gold, nanoparticles, polymers, condensation reagents, polysaccharides, polyamino acids, dendrimers, saponins, adsorption enhancers or fatty acids.

When the pharmaceutical composition is prepared, the pharmaceutical composition may be prepared using a commonly used dilutent or excipient, such as a lubricant, a sweetening agent, a flavoring agent, a suspension, a preservative, a filler, an extender, a binder, a wetting agent, a disintegrant, and a surfactant. A solid preparation for oral administration may comprise a tablet, a pill, a powder, a granule, a capsule, and the like, and the solid formulation may be prepared by mixing at least one excipient, for example, starch, calcium carbonate, sucrose or lactose, gelatin, and the like with the composition. Further, in addition to simple excipients, lubricants such as magnesium stearate and talc may also be used. Examples of a liquid preparation for oral administration comprise a suspension, a liquid for internal use, an emulsion, a syrup, and the like, and the liquid formulation may comprise, in addition to water and liquid paraffin which are simple commonly used diluents, various excipients, for example, a wetting agent, a sweetener, an aromatic, a preservative, and the like. A preparation for parenteral administration may comprise an aqueous sterile solution, a non-aqueous solvent, a suspension, an emulsion, a freeze-dried preparation, and a suppository. As the non-aqueous solvent and the suspension, it is possible to use propylene glycol, polyethylene glycol, a vegetable oil such as olive oil, an injectable ester such as ethyl oleate, and the like. As the base of the suppository, Witepsol, macrogol, Tween 61, cacao butter, laurin butter, glycerol gelatin, and the like may be used, and known diluents, excipients, and the like may be used when the composition is prepared in the form of a collyrium.

The pharmaceutical composition according to the present invention is administered in a pharmaceutically effective amount. In the present invention, “pharmaceutically effective amount” means an amount sufficient to treat diseases at a reasonable benefit/risk ratio applicable to medical treatment, and an effective dosage level may be determined according to factors comprising the type of disease of a patient, the severity of the disease, the activity of drugs, sensitivity to drugs, administration time, administration route, excretion rate, treatment period, and simultaneously used drugs, and other factors well known in the medical field.

The pharmaceutical composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this amount may be easily determined by a person with ordinary skill in the art to which the present invention pertains.

The pharmaceutical composition of the present invention may be administered to a subject in need via various routes. All methods of administration may be expected, but the pharmaceutical composition may be administered by, for example, oral administration, subcutaneous injection, peritoneal administration, intravenous injection, intramuscular injection, paraspinal space (intradural) injection, sublingual administration, buccal administration, intrarectal insertion, intravaginal injection, ocular administration, ear administration, nasal administration, inhalation, spraying via the mouth or nose, skin administration, transdermal administration, and the like.

The pharmaceutical composition of the present invention is determined by the type of drug that is an active ingredient, as well as various related factors such as the disease to be treated, the route of administration, the age, sex, and body weight of a patient, and the severity of the disease.

As used herein, the “subject” refers to a subject in need of treatment of a disease, and more specifically, refers to a mammal such as a human or a non-human primate, a mouse, a rat, a dog, a cat, a horse, and a cow.

“Administration” as used herein refers to the provision of a predetermined composition of the present invention to a subject in need thereof by any suitable method.

In the present invention, prevention refers to all actions of suppressing or delaying the onset of a target disease, and treatment refers to all actions which the target disease and its associated metabolic disorders are ameliorated or beneficially altered by administration of the pharmaceutical composition according to the present invention.

As yet another aspect of the present invention, the present invention may provide a method for preparing a stem cell genetically engineered to overexpress a carcinoembryonic antigen-related cell adhesion molecule (CEACAM) family protein, the method comprising (a) cloning a viral vector expressing a CEACAM family protein; (b) preparing a virus comprising the vector; and (c) overexpressing the CEACAM family protein by infecting stem cells with the virus prepared in Step (b).

In the present invention, the viral vector may be one or more selected from the group consisting of a lentiviral vector, a retroviral vector, an adenoviral vector, and a paramyxovirus vector, but is not limited thereto.

As used herein, the term vector refers to a means for expressing a target gene in a host cell. The vector comprises, for example, a viral vector such as a plasmid vector, a cosmid vector, a bacteriophages vector, and an adeno-associated viral vector, in addition to the vectors listed above. A vector that may be used as the recombinant vector may be produced by manipulating a plasmid (for example, pSC101, pGV1106, pACYC177. ColE1, pKT230, pME290, pBR322, pUC8/9, pUC6, pBD9, pHC79, pU61, pLAFR1, pHV14, pGEX series, pET series, pUC19, and the like), a phage or a virus (for example, SV40, and the like) which is commonly used in the art.

A polynucleotide encoding the protein complex in the recombinant vector may be operably linked to a promoter. The term operatively linked means a functional linkage between a nucleotide expression regulatory sequence (for example, a promoter sequence) and another nucleotide sequence. The regulatory sequence can be operatively linked to regulate the transcription and/or translation of other nucleotide sequences.

The recombinant vector can typically be constructed as a vector for cloning or a vector for expression. As the expression vector, a typical vector used to express a foreign protein in a plant, animal or microorganism in the art may be used. The recombinant vector may be constructed by various methods known in the art.

Terms or words used in the specification and the claims should not be interpreted as being limited to typical or dictionary meanings and should be interpreted with a meaning and a concept that are consistent with the technical spirit of the present invention based on the principle that an inventor can appropriately define the concept of a term in order to describe his/her own invention in the best way.

Hereinafter, preferred examples for helping with understanding of the present invention will be suggested. However, the following examples are provided only so that the present invention may be more easily understood, and the content of the present invention is not limited by the following examples.

### EXAMPLES

Experimental Materials and Methods

1. Production of Lentivirus for CEACAM1 Expression

To produce a lentivirus for overexpression of a CEACAM1 protein, cDNA sequences for four CEACAM1 isoforms were synthesized by commissioning Integrated DNA Technologies, USA. Information on the cDNA sequences and the proteins encoded by the same are set forth in the following Table 1 and Sequence Listing.

TABLE 1

| Name | NCBI Reference No. | Type | Sequence Listing No. |
|---|---|---|---|
| CEACAM1-4L | NM_001712.5 | DNA | SEQ ID NO: 1 |
| | | Protein | SEQ ID NO: 2 |
| CEACAM1-4S | NM_001024912.2 | DNA | SEO ID NO: 3 |
| | | Protein | SEQ ID NO: 4 |
| CEACAM1-3L | NM_001184813.1 | DNA | SEQ ID NO: 5 |
| | | Protein | SEQ ID NO: 6 |
| CEACAM1-3S | NM_001184816.1 | DNA | SEQ ID NO: 7 |
| | | Protein | SEQ ID NO: 8 |

The four CEACAM1 cDNA sequences were inserted between the XbaI and EcoRI restriction enzyme sites of the pCDH-CMV-EF1α-GFP-Puro (System Biosciences. USA) plasmid. A 293TN cell line (System Biosciences, USA) was cultured in a DMEM medium (Dulbecco's Modified Eagle Medium: Gibco®, Thermo Fisher Scientific, USA) supplemented with 10% FBS (Gibco®), 1% GlutaMax (Gibco®) and 1% penicillin/streptomycin (Gibco®). After the 293TN cell line was cultured in a 6-well plate for 16 hours, a total of 2.22 μg of a plasmid mixed with pCDH-CMV-EF1α-GFP-Puro in which CEACAM1 was cloned, psPAX2 (Addgene, USA) and pMD2.G (Addgene) was mixed with X-tremeGENE 9 (Roche, Switzerland) according to the manufacturer's instructions, and then the 293TN cell line was treated with the resulting mixture and cultured in an environment of 37° C. and 5% CO₂ for 24 hours. Thereafter, the cell line was further cultured for 2 days while exchanging the medium with a new medium. After the culture solution was collected and centrifuged at 1,500 rpm for 3 minutes at −2 to −8° C., the supernatant was collected, filtered through a 0.45 μm standard filter, and then transferred to a new tube. To measure the viral titer, a vims culture solution diluted to a concentration of 10⁻¹ to 10⁻⁵ was inoculated into the 293TN cell line, and 48 hours later, GFP-expressing cells were analyzed by flow cytometry. The viral titer was calculated by the following method:

Viral titer(Transduction Unit/mL,TU/mL)=number of cells x % GFP×dilution factor/inoculum volume

2. Production of CEACAM1-Overexpressing Stem Cells

Umbilical cord-derived mesenchymal stem cells (UC-MSCs) were used as stem cells for CEACAM1 overexpression. UC-MSCs were cultured in an MEM-α (Gibco®) medium supplemented with 10% FBS (Gibco®), 1% GlutaMax (Gibco®) and 1% penicillin/streptomycin (Gibco®) for 3 to 4 days. After culturing, the UC-MSCs were gently harvested from a culture vessel using Accutase (Innovative Cell Technologies, USA) and seeded in a T25 flask (Corning, USA) the day before lentivirus inoculation. The next day, the UC-MSCs were infected with a lentivirus at a multiplicity of infection (MOI) of 4 together with 10 μg/ml polybrene (Sigma, USA) for 48 hours. Thereafter, the culture solution was removed and the UC-MSCs were cultured in a culture solution comprising 2.5 μg/ml puromycin (Merck, USA) for 8 days. CEACAM1 expression was confirmed by cell surface staining or reverse transcription-polymerase chain reaction (RT-PCR).

3. Confirmation of Protein and RNA Expression

In order to confirm cell surface expression, the Fc receptors of the UC-MSCs were incubated with an Fe receptor binding inhibitor polyclonal antibody (Invitrogen, USA) at 4° C. for 15 minutes. Thereafter, the Fe receptors of the UC-MSCs were incubated with PE-CEACAM1 (R&D Systems, USA) at room temperature in a dark room for 30 minutes, washed twice with fluorescence activated cell sorter (FACS) buffer, and then analyzed by a flow cytometer.

To confirm isoform-specific RNA expression of CEACAM1, UC-MSCs were isolated with Trizol RNA isolation reagent (Invitrogen) according to the manufacturer's instructions. Then, after 1 μg of RNA was synthesized into cDNA using a ReverTraAce™ qPCR-RT kit (TOYOBO, Japan), a polymerase chain reaction was performed by repeating 30 cycles under the conditions of 1 minute at 94° C., 45 seconds at 94° C., 45 seconds at 64° C., 10 minute at 72° C., and 10 minutes at 72° C. using the primers in the following Table 2. To confirm the size of the reaction product, the reaction product was electrophoresed on a 4% UltraPure™ agarose gel (Invitrogen).

TABLE 2

| Primer | Direction | Sequence | Sequence Listing No. |
|---|---|---|---|
| CEACAM1-F | Forward | NM_001712.5 | SEQ ID NO: 9 |
| CEACAM1-L | Reverse | NM_001024912.2 | SEQ ID NO: 10 |
| CEACAM1-S | Reverse | NM_001184813.1 | SEQ ID NO: 11 |

4. Confirmation of CEACAM1 Expression by Cell

An NK92 cell line (ATCC, USA) was cultured in an MEM-α (Gibco®) medium supplemented with 20% FBS (Gibco®), a 1% aqueous vitamin solution, 1% penicillin/streptomycin (Gibco®), and 0.1 mM 2-mercaptoethanol (Gibco®).

Cryopreserved peripheral blood mononuclear cells (PBMCs) were thawed and placed in an RPVI1640 (Gibco®) medium supplemented with 10% FBS (Gibco®), 1% Glutamax™ (Gibco®) and 1% penicillin/streptomycin (Gibco®). Cryopreserved peripheral blood mononuclear cells (PBMCs) were cultured in a medium to which 20 μg/ml Phytohemagglutinin P (PHA-P; Sigma), 10 ng/ml IL-12p70 (Peprotech, USA), 200 Unit/ml IL-2 (Roche), 10 ng/ml IL-15 (Peprotech) or 10 ng/ml IFNγ (Peprotech) was added under 37° C. and 5% CO₂ conditions for 2 to 3 days. The NK92 cell line or pre-activated PBMCs were harvested and washed once with FACS buffer, and then the Fc receptors of the cells were incubated with an Fe receptor binding inhibitor polyclonal antibody (Invitrogen) at 4° C. for 15 minutes. Then, the cells were incubated at room temperature in the dark for 30 minutes using FITC-CD56 (NCAM16.2; BD Biosciences, USA), PE-CEACAM1 (283340; R&D Systems), and PerCP-CD3 (SK7; BD Biosciences) as cell surface markers. The cells were washed twice with FACS buffer, and then analyzed by a flow cytometer.

5. Cytotoxicity Activity Analysis of NK Cells

UC-MSCs cultured for 3 to 4 days were detached from the culture vessel, and then UC-MSCs were labeled at 37° C. for 30 minutes by adding a BATDA labeling reagent (PerkinElmer, USA) at a final concentration of 40 LM. BATDA-labeled UC-MSCs and expanded NK cells (ENK cells) or PBMCs pre-activated with PHA and IL-2 for 2 days were both put into a 96-well V bottom plate, and were cultured in an incubator in an environment of 37° C. and 5% CO₂ for 2 hours (ENK cells) or 4 hours (pre-activated PBMCs). Alternatively, BATDA-labeled UC-MSCs and Expanded NK cells (ENK cells) or PBMCs pre-activated with PHA and IL-2 for 2 days were cultured with the NK92 cell line for 3 hours. Thereafter, the culture solution was harvested through centrifugation (1500 rpm, 3 minutes), and the TDA in the culture solution discharged through cell lysis was added to a DELFIA Eu-Solution (PerkinElmer) fluorescent dye. The degree of fluorescence was analyzed by a microplate reader (Ex/Em, 340 nm/615 un), and the degree of specific lysis was calculated by the following method.

$$\% Specific lysis = \frac{Experimental release ( counts ) - Spontaneous release ( counts )}{Maximum release ( counts ) - Spontaneous release ( counts )} \times 100$$

6. Degranulation Activity Analysis of NK Cells

After NK cytotoxic activity analysis of ENK cells or pre-activated PBMCs, Fe receptors were incubated with an Fc receptor binding inhibitor polyclonal antibody (Invitrogen) at 4° C. for 15 minutes in order to perform the degranulation activity analysis of remaining cells. Thereafter, cell surface markers were incubated with FITC-CD107a (H4A3; BD Biosciences), PE-CD56 (NCAM16.2; BD Biosciences), and PerCP-CD3 (SK7; BD Biosciences) in a dark room at 4° C. for 30 minutes. The cells were washed twice with FACS buffer, and then analyzed by a flow cytometer.

7. T Cell Proliferation Suppression Assay

Cryopreserved PBMCs were thawed and placed in a RPMI1640 medium supplemented with 10% FBS, 1% Glutamax™ (Gibco®), and 1% penicillin/streptomycin, and allowed to rest for 3 hours. Thereafter, PBMCs were harvested and labeled using a 1 μM CellTrace™ carboxyfluorescein succinimidyl ester (CFSE) Cell Proliferation Kit by incubation in a constant temperature water bath at 37° C. for 10 minutes. A 10-fold volume of RPMI1640 medium was added thereto, and the resultant was centrifuged (1400 rpm, 5 minutes) and washed three times. MSCs cultured for 3 to 4 days were detached from the culture vessel and cultured with 20 μg/mL PHA-P (Sigma) at a ratio of 1:5 (MSC:PBMC) in an incubator in an environment of 37° C. and 5% CO₂ for 5 days. Thereafter, the cultured cells were washed once with FACS buffer, and then the Fc receptors of the cells were incubated with an Fe receptor binding inhibitor polyclonal antibody (Invitrogen) at 4° C. for 15 minutes. Thereafter, CD4 T cells were labeled by incubation with PE-CD25 (M-A251; BD Biosciences), PerCP-CD3 (SK7; BD Biosciences), and APC-CD4 (SK3; 10 BD Biosciences) markers in a dark room at 4° C. for 30 minutes, and CD8 T cells were labeled by incubation with PE-CD25 (M-A251; BD Biosciences), PerCP-CD3 (SK7; BD Biosciences), and APC-CD8 (SK1; BD Biosciences) markers in a dark room at 4° C. for 30 minutes. Thereafter, the cells were washed twice with FACS buffer, and then analyzed by a flow cytometer.

8. Method for analyzing T cell activity and ability to produce IFNγ and TNFα

The PBMCs of a healthy person were activated and cultured with 5 μg/mL PHA-P (Sigma) and 200 unit/mL IL-2 (Roche) for 3 days. MSCs were seeded at 2×10⁴ cells each in 96-well flat bottom plates (SPL), and the next day, 1-10⁵ activated PBMCs were cultured for 1 hour with 1×10 P815 cells coated with 10 μg/mL of an anti-CD3 antibody (eBioscience, OKT3) and an anti-CD28 antibody (eBioscience, CD28.2), respectively. Then, Golgistop (BD) and Golgiplug (BD) were added, the resultant was cultured for additional 2 hours, and then the cells were harvested and washed once with FACS buffer. The Fc receptors of the cells were incubated with an Fc receptor binding inhibitor polyclonal antibody (Invitrogen) at 4° C. for 15 minutes to prevent non-specific binding of antibodies. Thereafter, CD4 T cells were labeled by incubation with PerCP-CD3 (BD Biosciences, SK7) and APC-CD4 (BD Biosciences, SK3) markers in a dark room at 4° C. for 30 minutes, and CD8 T cells were labeled by incubation with PerCP-CD3 (BD Biosciences, SK7) and PE/Cy7-CD8 (BD Biosciences, SKI) markers in a dark room at 4° C. for 30 minutes. Thereafter, the cells were washed twice with FACS buffer, incubated with Fix/Perm buffer (BD Biosciences) in a dark room at 4° C. for 20 minutes, and then supplemented and washed twice with Perm/Wash buffer (BD Biosciences). For intracellular cytokine detection, the cells were incubated with FITC-IFNγ(BD, 25723.11) and PE-TNFα (eBioscience, MAb11) in a dark room at 4° C. for 12 hours or more, washed twice with Penn/Wash buffer, then washed twice with FACS buffer, and then analyzed by a flow cytometer.

9. Graft-Versus-Host Disease (GvHD) Induction in Animal Model

Seven-week-old male NOD-scid IL2Rgamna^{mall} (NSG) were purchased from The Jackson Laboratory (#005557) and then allowed to acclimate for two weeks. Then, the animals were irradiated with 2.0 Gy using an X-RAD 320 x-ray irradiator (Precision X-Ray Inc.), and the next day, 2.5×10⁶ healthy human-derived PBMCs (STEMCELL Technology) or PBS were intravenously injected. On day 18 after PBMC injection, 5×10⁵ of MEMα or empty-MSCs, CEACAM1-4L-overexpressing MSCs, or CEACAM1-4S-overexpressing MSCs supplemented with PBS or 0.5% FBS were intravenously injected into each of 5 animals, and body weight was measured at 3-day intervals for 60 days.

10. Production of MSCs for In Vivo Injection into Animal Model

In order to produce CEACAM1-expressing MSCs for in vivo injection into an animal model, MSCs were gently harvested from the culture vessel using Accutase (Innovative Cell Technologies) and then cultured in a T25 flak (Corning) the day before lentivirus inoculation. The next day, a CEACAM1-expressing lentivirus was inoculated at an MOI of 4 with 10 μg/mL polybrene (Sigma) for 24 hours, and then 10 the resultant was cultured for additional 24 hours after replacing the culture solution with a new culture solution. The cells were detached with Accutase, and then were additionally cultured in a T175 (Thermo) or T75 (Corning) flask at a scale of 2,000 cells/cm² for 5 days. MSCs were washed twice with DPBS (Gibco®), reacted with Accutase supplemented with 2 mM EDTA at 37° C. for 5 minutes, detached, and then filtered through a 40 μm strainer and collected. Cells were collected by centrifugation at 1,000 rpm for 5 minutes and then washed twice with an MEMα culture solution supplemented with 10% FBS. Thereafter, MSCs were released with MEMα supplemented with 0.5% FBS, filtered through a 70 μm strainer, and then prepared at 5×10⁵ cells/200 pd.

### Example 1. Analysis of CEACAM1 Expression Pattern in Stem Cells

Since normal stem cells comprising umbilical cord-derived mesenchymal stem cells (UC-MSCs) do not express CEACAM1 under normal circumstances, expression was induced using a lentivirus as described in the “Experimental materials and methods” section above in order to stably overexpress CEACAM1. It was confirmed by GFP whether the stem cells were infected with the lentivirus, and the cell surface expression of CEACAM1 was confirmed using a PE-CEACAM1 antibody.

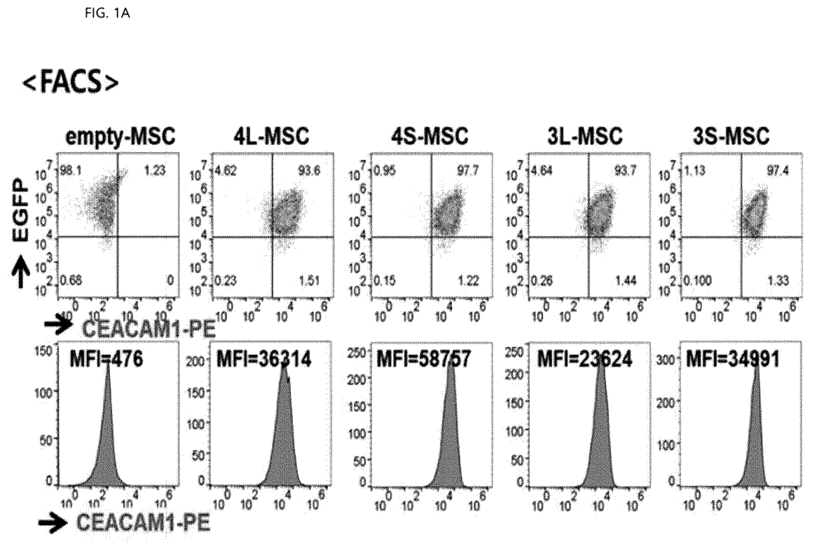

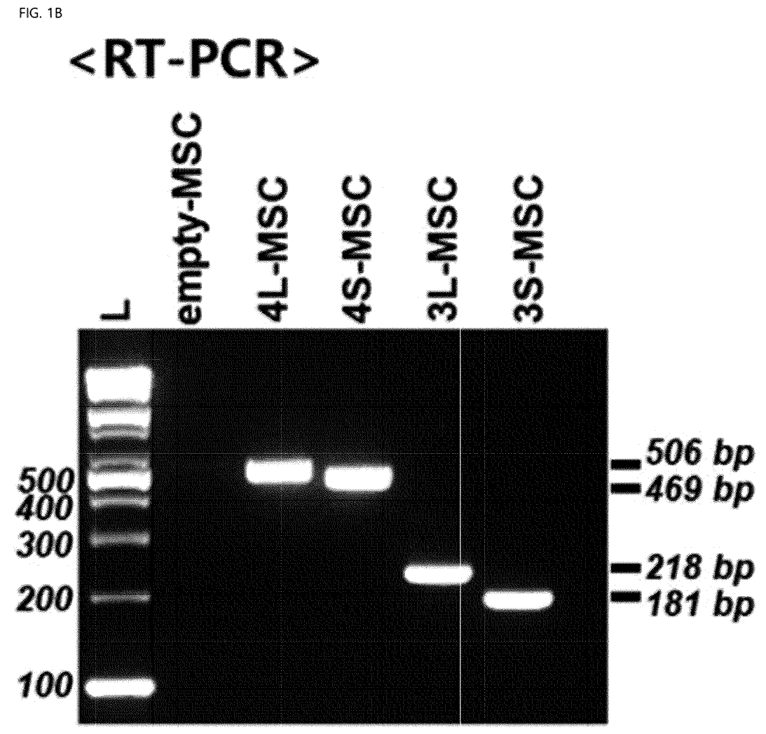

The results are illustrated in FIGS. 1A and 1B. First, CEACAM1 expression was shown to be negative in mesenchymal stem cells (empty-MSCs) infected with a lentivirus comprising an empty vector, and positive expression was confirmed in all CEACAM1-overexpressing MSCs. When the expression levels were compared by isoform, CEACAM1-4 was higher than CEACAM1-3, and the long form was lower than the short form (FIG. 1A). Such a trend suggests the possibility that different CEACAM1 isoforms have different characteristics.

Meanwhile, since the PE-CEACAM1 antibody is not capable of isoform-specific discrimination. CEACAM1 mRNA species were analyzed by reverse transcription-polymerase chain reaction to further verify isoform expression. As a result, the size of the normal PCR reaction product for the intentionally introduced CEACAM1 isoforms was confirmed (FIG. 1B). These results verify the feasibility of lentiviral-mediated CEACM1 isoform-specific gene transfer into UC-MSCs.

### Example 2. Confirmation of CEACAM1 Expression Pattern of PBMC According to Cytokines

In order to investigate the expression of CEACAM1 in NK cells, natural killer T (NKT) cells, and T cells, which are representative immune cells in PBMCs, the stem cells were cultured in media comprising PHA-P or comprising IL-12, IL-2, IL-15 or IFNγ and PHA-P for 3 days as described in Section 4 of ‘Experimental materials and methods.’ NK cells, NKT cells, and T cells were analyzed by gating with CD56⁺CD3ε⁻, CD56+CD3ε⁺, and CD56⁻CD3ε⁺, respectively, and compared with the level of CEACAM1 expression before activation.

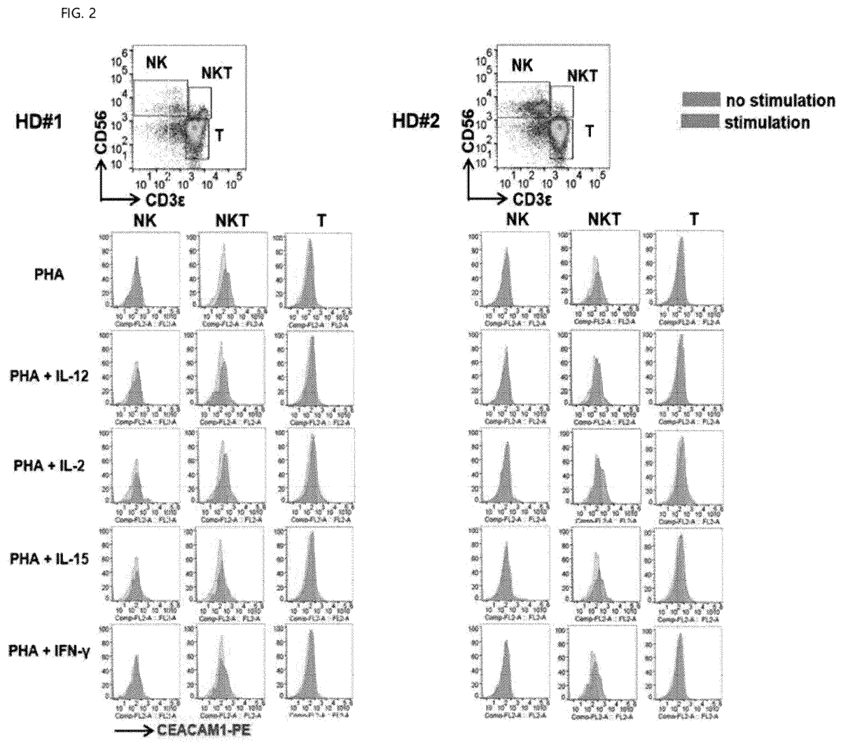

As a result, as can be seen in FIG. 2, although it was confirmed that CEACAM1 expression was increased under conditions of not only PHA-P alone but also various combinations of cytokines and PHA-P, CEACAM1 expression was induced the most under PHA+IL-2 and PHA+IL-15 culture conditions. As a result of analyzing CEACAM1 expression patterns in each type of cell, CEACAM1 expression was increased the most in NKT cells, and CEACAM1 expression was higher in T cells than in NK cells. Such a trend suggests that CEACAM1 is expressed at various levels according to immune cells and may be involved in regulating the immune response of the corresponding immune cells.

### Example 3. Confirmation of ENK Cytotoxic Resistance of CEACAM1-Over Expressing Stem Cells

To confirm whether the overexpression of CEACAM1 in UC-MSCs can evade the cytotoxic activity of NK cells, empty-MSCs or four types of CEACAM1-overexpressing MSCs were cultured with ENK cells for 2 hours as described above.

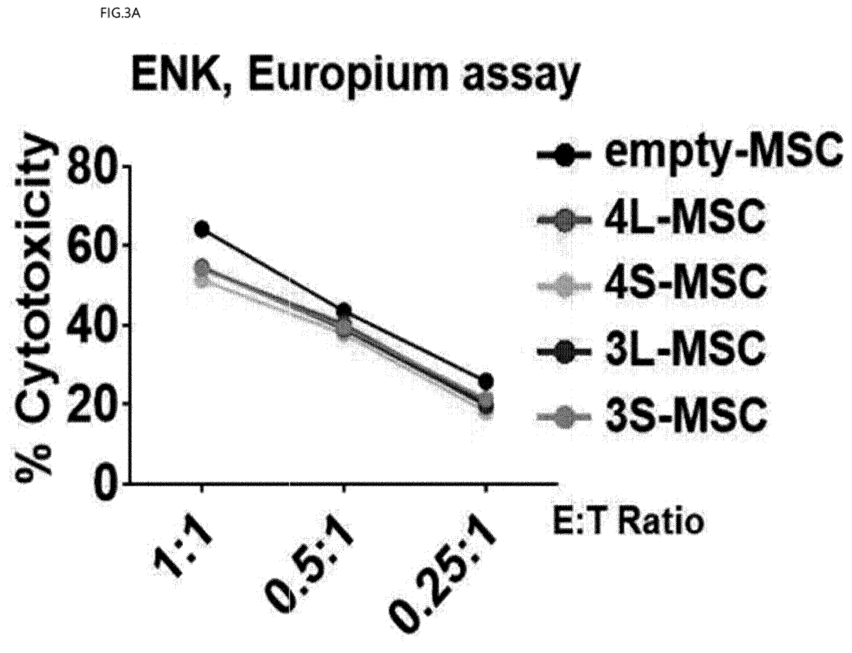

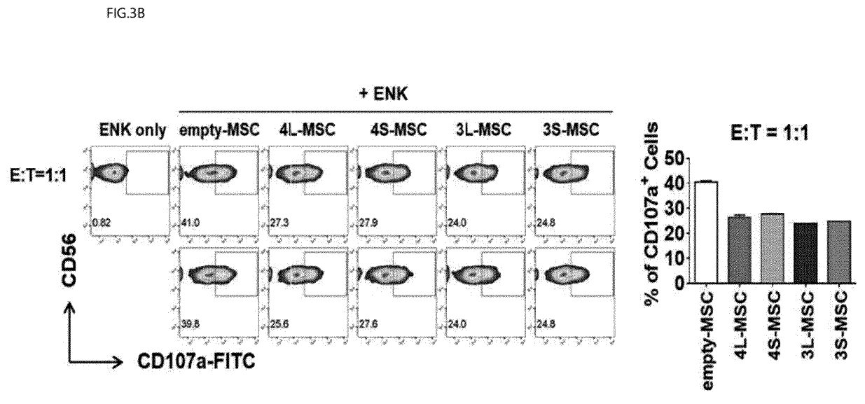

As a result of measuring the degree of cell lysis by a culture solution, it was confirmed that the survival of CEACAM1-overexpressing UC-MSCs was increased (FIG. 3A). These results suggest that UC-MSCs have resistance to ENK cells due to CEACAM1. In addition, as a result of analyzing the degranulation activity of ENK cells in terms of CD107a expression, it was confirmed that CD107a expression was decreased in CEACAM1-overexpressing UC-MSCs (FIG. 3B). This means that the cytotoxic ability of ENK cells was reduced due to CEACAM1 of UC-MSCs. These results demonstrate that CEACAM1, which is expressed in stem cells, is an important factor in evading the immune response of ENK cells.

### Example 4. Confirmation of NK Cytotoxic Resistance of CEACAM1-Overexpressing Stem Cells in Peripheral Blood Mononuclear Cells (PBMCs)

Empty-MSCs or CEACAM1-overexpressing MSCs were cultured with PBMCs pre-activated for 2 days for 4 hours as described in the “Experimental materials and methods” section.

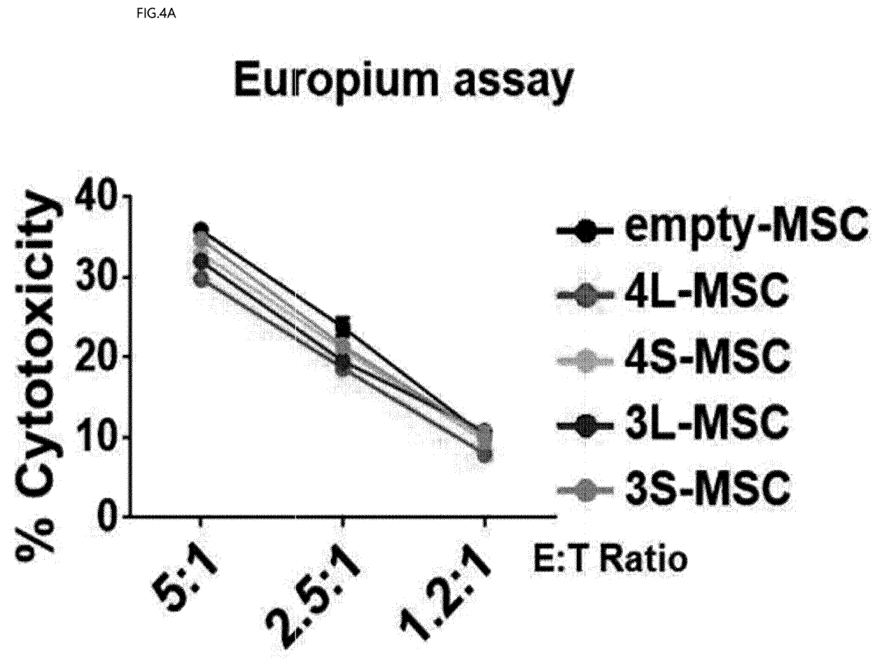

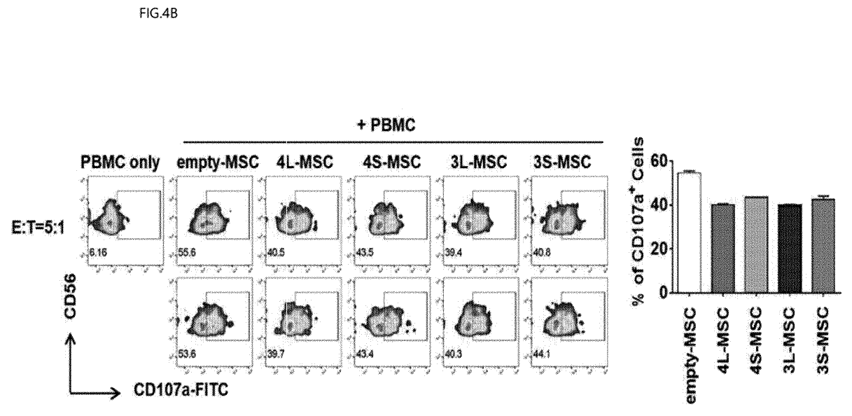

As a result of measuring the degree of cell lysis by a culture solution, it was confirmed that the survival of CEACAM1-overexpressing UC-MSCs was increased (FIG. 4A). In addition, as a result of analyzing the degranulation activity of NK cells in terms of CD107a expression, it was confirmed that CD107a expression was decreased in CEACAM1-overexpressing UC-MSCs (FIG. 4B). This means that the cytotoxic ability of NK cells was reduced due to CEACAM1 of UC-MSCs. These results confirmed that CEACAM1, which is expressed in MSCs, is an important factor in evading the immune response of NK cells. That is, these results suggest that NK cells in PBMCs affect MSC cell death and CEACM1 expression is a key factor in evading the same.

### Example 5. Confirmation of Cytotoxic Resistance of CEACAM1-Overexpressing Stem Cells by NK92 Cell Line

As a result of confirming the expression level of CEACAM1 in the NK92 cell line, the expression level was higher than that of other normal immune cells comprising NK cells.

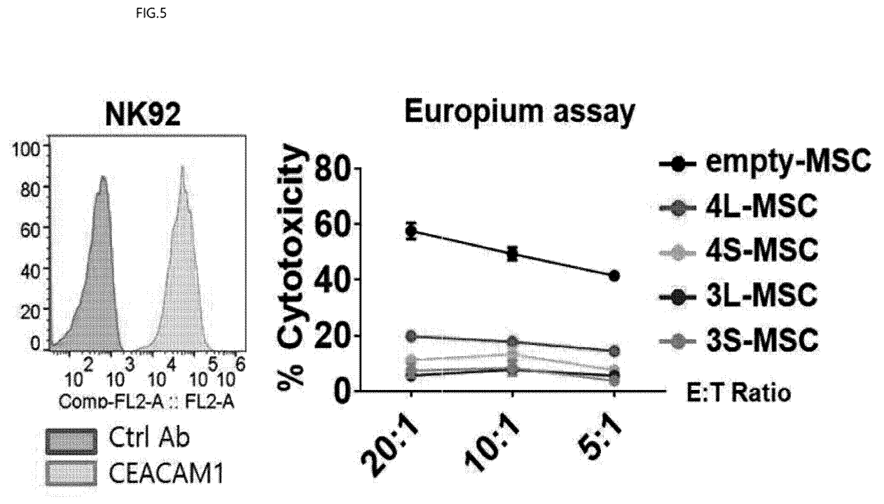

As a result of comparing the cytotoxicity against empty-MSCs or CEACAM1-overexpressing MSCs as described in the “Experimental materials and methods” section, the cell killing ability against CEACAM1-overexpressing MSCs was decreased (FIG. 5). From the experimental results using ENK cells, pre-activated PBMCs and NK92 cell lines, it can be seen that the interaction between CEACAM11 of NK cells and CEACAM1 of MSCs reduces the ability of NK cells to kill MSCs.

### Example 6. Confirmation of Ability of CEACAM1-Overexpressing Stem Cells to Suppress Proliferation of CD4 T Cells and CD8 T Cells

Through screening of CEACAM1 expression in PBMCs, it was confirmed that the expression of CEACAM1 was also induced in T cells by various stimuli such as cytokines. To confirm whether CEACM1-overexpressing MSCs can suppress T cell activity, PBMCs were labeled with CFSE as described in Section 7 of “Experimental materials and methods,” and were cultured with PHA-P and MSCs for 5 days.

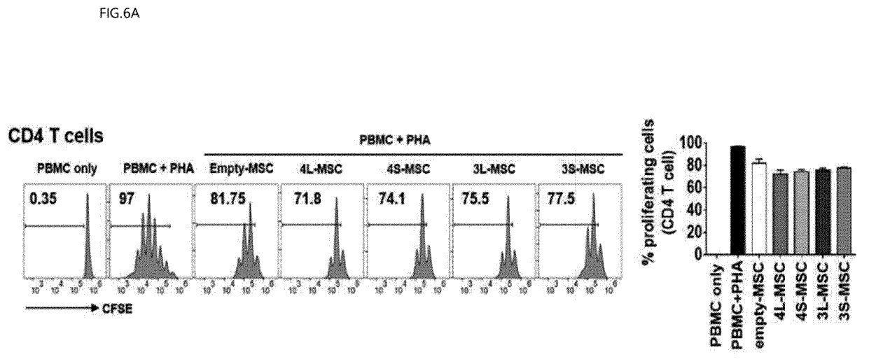

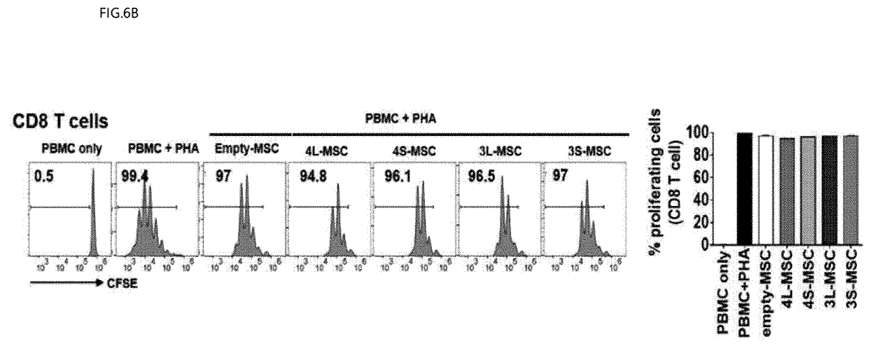

As a result of analyzing the proliferation rate of CD4 T cells by a flow cytometer, CD4 T cells proliferated, on average, by 97% with PHA-P, by 81.75% when cultured with empty-MSCs, and by 74.7% when cultured with CEACAM1-overexpressing MSCs (FIG. 6A). These results indicate that the proportion of non-dividing CD4 T cells due to empty-MSCs was 15.3% and the proportion of non-dividing CD4 T cells due to CEACM1-overexpressing MSCs was 22.3%, indicating that CEACM1 is involved in the suppression of CD4 T cell division.

Meanwhile, as a result of analyzing the proliferation rate of CD8 T cells by a flow cytometer, it was confirmed that CD8 T cells proliferated, on average, by 99.4% with PHA-P, by 97% when cultured with empty-MSCs, and by 96.1% when cultured with CEACAM1-overexpressing MSCs (FIG. 6B). In the case of CD8 T cells, most of the CD8 T cells proliferate even in the presence of empty-MSCs or CEACM1-overexpressing MSCs, but when the degree of CFSE dilution was compared, it is determined that the proliferation suppression ability of CEACAM1-overexpressing MSCs is observed. Overall, these results indicate that CEACAM1-overexpressing MSCs are more dominant in suppressing CD14 T cell proliferation than CD8 T cell proliferation.

### Example 7. Re-Confirmation of Ability of CEACAM1-Overexpressing Stem Cells to Suppress Proliferation of CD4 T Cells and CD8 T Cells

To closely compare the proliferation suppression ability of empty-MSCs and CEACM1-expressing MSCs, the CFSE fluorescence intensity of CD4 T or CD8 T cells was comparatively analyzed.

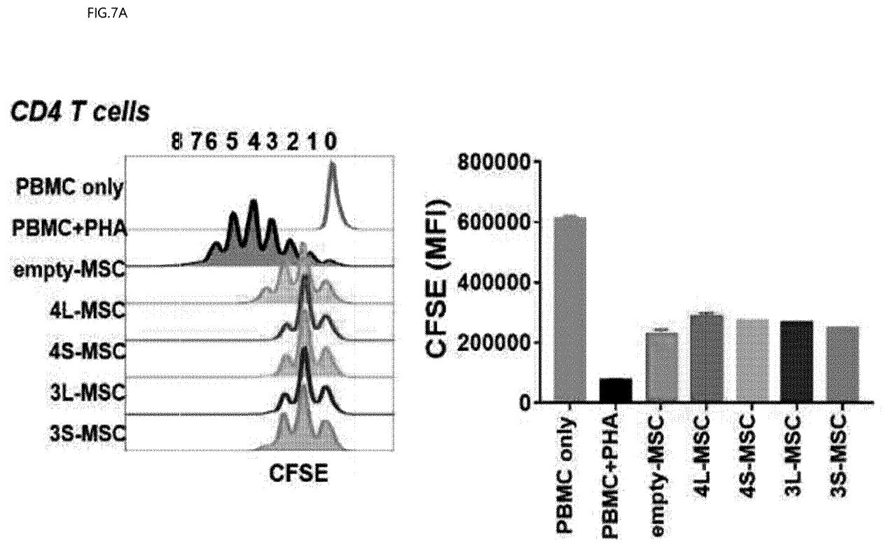

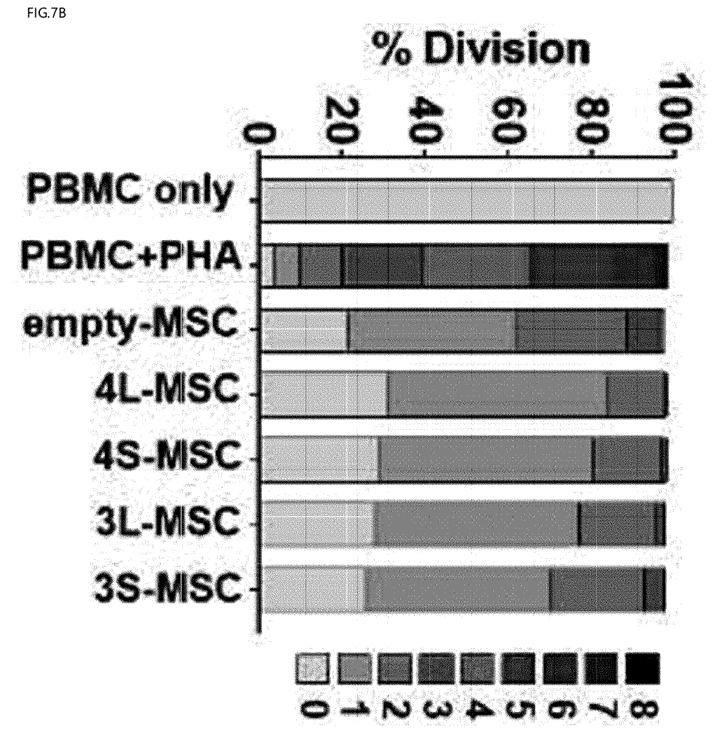

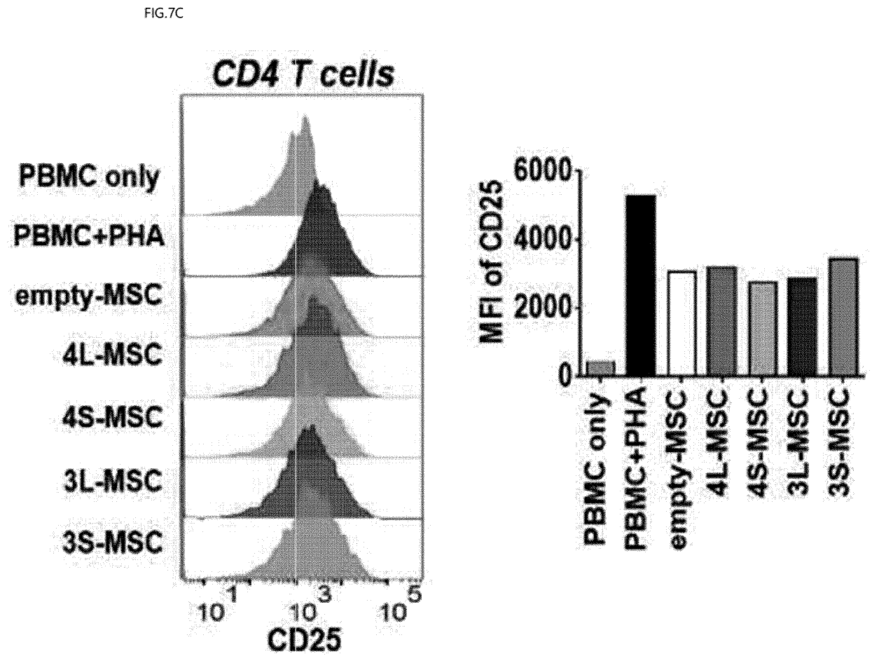

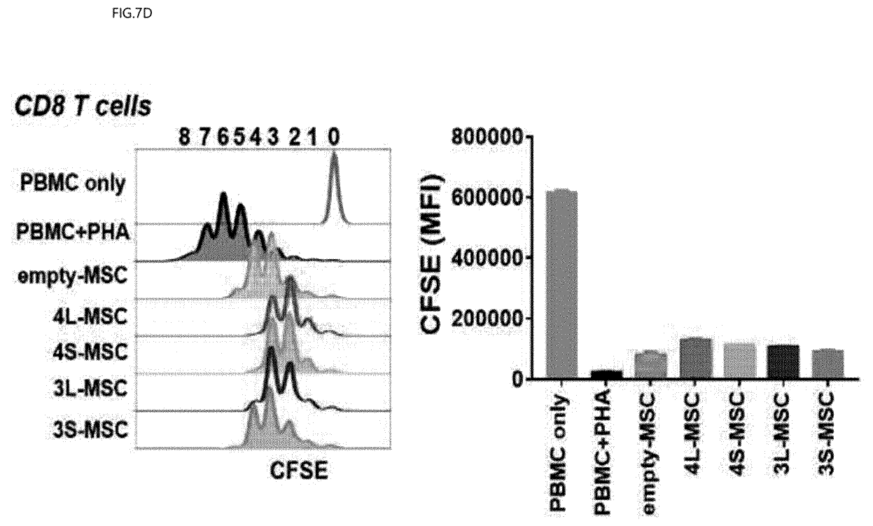

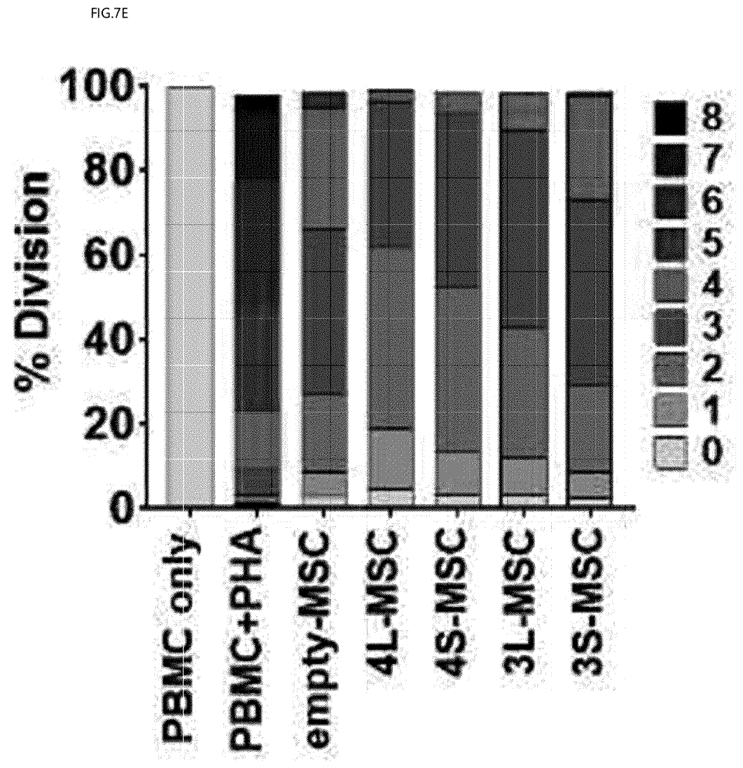

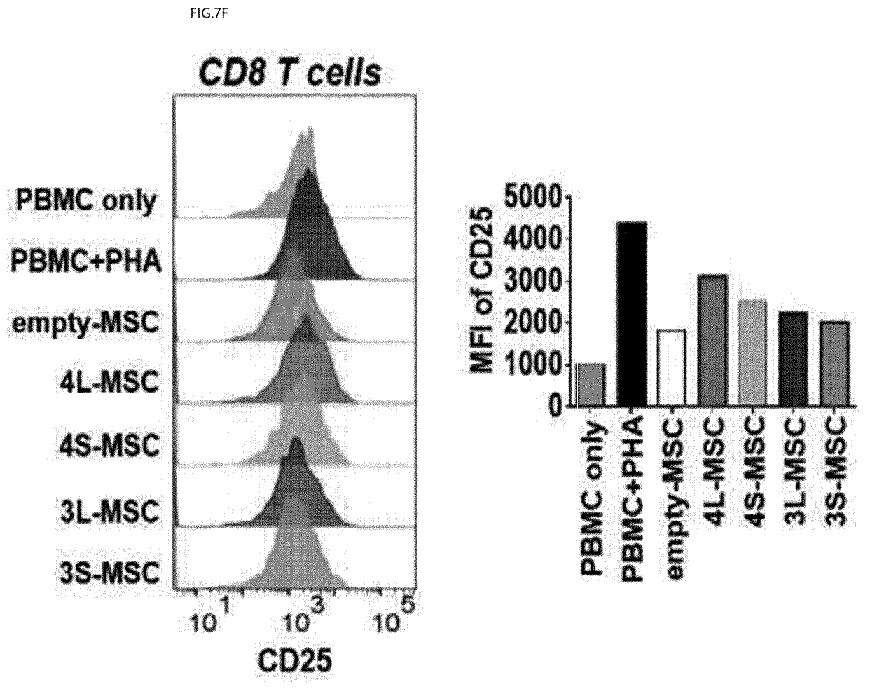

First, during PHA-P-stimulated CD4 T or CD8 T cell division, CFSE fluorescence intensity was decreased, and division was suppressed by empty-MSCs, resulting in an increase in fluorescence intensity. It was shown that when CD4 T or CD8 T cells were cultured with CEACM1-overexpressing MSCs, the CFSE fluorescence intensity of CD4 T or CD8 T cells was further increased than when CD4 T or CD8 T cells were cultured with empty-MSCs (FIGS. 7A and 7D). These results indicate that CEACM1-overexpressing MSCs are superior to empty-MSCs in overall T cell proliferation suppression ability.

For a more in-depth analysis, the number and degree of division of CD4 T cells by MSCs were measured. As a result of analyzing the 0th (undivided) division, it was found to be 21% when CD4 T cells were cultured with empty-MSCs and 32% when CD4 T cells were cultured with CEACAM1-4L-overexpressing MSCs, and as a result of analyzing the first division, it was found to be 40% when CD4 T cells were cultured with empty-MSCs and 53% when CD4 T cells were cultured with CEACAM1-4L-overexpressing MSCs. As a result of analyzing the second division, it was found to be 27% when CD4 T cells were cultured with empty-MSCs and 13% when CD4 T cells were cultured with CEACAM1-4L-overexpressing MSCs (FIG. 7B). These results indicate that the division of CD4 T cells cultured with CEACAM1-overexpressing MSCs is suppressed from the initial stage of division.

The number and degree of division of CD8 T cells were measured by the same method. As a result of analyzing the first division, it was found to be 6% when CD8 T cells were cultured with empty-MSCs and 14% when CD8 T cells were cultured with CEACAM1-4L-overexpressing MSCs. As a result of analyzing the second division, it was found to be 18% when CD8 T cells were cultured with empty-MSCs and 43% when CD8 T cells were cultured with CEACAM-4L-overexpressing MSCs. As a result of analyzing the third division, it was found to be 39% when CD8 T cells were cultured with empty-MSCs and 34% when CD8 T cells were cultured with CEACAM1-4L-overexpressing MSCs. As a result of analyzing the fourth division, it was found to be 29% when CD8 T cells were cultured with empty-MSCs and 3% when CD8 T cells were cultured with CEACAM1-4L-overexpressing MSCs (FIG. 7E). It can be seen that CD8 T cells cultured with empty-MSCs were divided up to the third round and the division was suppressed from the fourth round, whereas CD8 T cells cultured with CEACAM1-overexpressing MSCs were divided up to the second round and the division was suppressed from the third round. These results indicate that CEACM1-overexpressing MSCs have better ability to suppress the proliferation of CD8 T cells than empty-MSCs. However, as a result of analyzing CD25, which is an activity indicator of T cells, there was no significant difference between CEACAM1 isoforms (FIGS. 7C and 7F).

### Example 8. Confirmation of Ability of CEACAM1-Overexpressing Stem Cells to Relate T Cell Cytokine Secretion

As it was confirmed that CEACAM1-overexpressing stem cells could suppress the proliferation of CD4 T cells and CD8 T cells through the above examples, it was confirmed whether CEACAM1-overexpressing stem cells also affect the ability of T cells to secrete cytokines. For this purpose, PBMCs activated by PHA-P and IL-2 for 3 days were cultured with MSCs and P815 cells coated with an anti-CD3 antibody and anti-CD28 antibody for 3 hours, and intracellular IFNγ and TNFα of CD4 and CD8 T cells were analyzed by fluorescent staining.

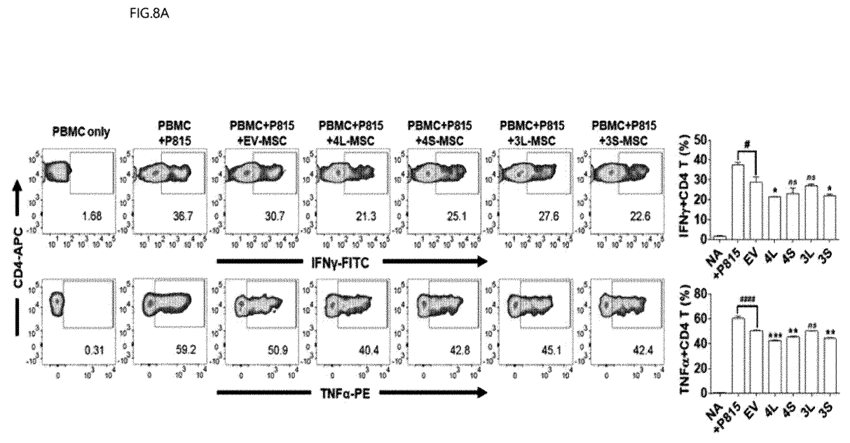

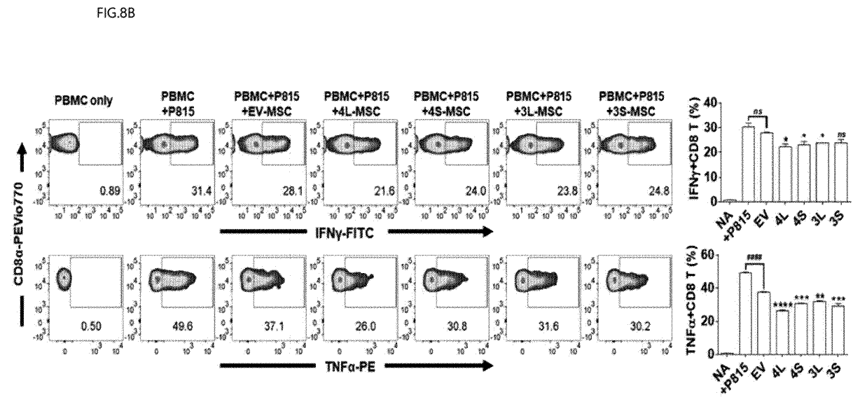

First, as a result of analyzing the IFNγ and TNFα secretion patterns of CD4 T cells cultured with CEACAM1-overexpressing stem cells, CEACAM1-4L-overexpressing MSCs were statistically the most effective in suppressing cytokine secretion. Empty-MSCs decreased IFN7 secretion of CD4 T cells by 23.5%, whereas the IFNγ secretion was decreased by 42.8% when CD4 T cells were cultured with CEACAM1-4L-overexpressing MSCs. In addition, empty-MSCs decreased TNFα secretion by 16.4%, whereas CEACAM1-4L-overexpressing MSCs decreased TNFα secretion by 29.8% (FIG. 8A). Subsequently as a result of analyzing the IFNγ and TNFα secretion patterns of CD8 T cells cultured with CEACAM1-overexpressing stem cells, empty-MSCs decreased the IFNγ secretion of CD8 T cells by 7.78%, whereas CEACAM1-4L-overexpressing MSCs decreased the IFNγ secretion by 25.9%. In addition, empty-MSCs decreased TNFα secretion by 23.8%, whereas CEACAM1-4L-overexpressing MSCs decreased TNFα secretion by 46.4% (FIG. 8B). These results indicate that MSCs suppress the cytokine secretion of T cells, and suggest that the suppressive ability is further enhanced by CEACAM1-4L overexpression.

Example 9. Confirmation of Therapeutic Effect of CEACAM1-Overexpressing Stem Cells on Raft-Versus-Host Disease

As it was confirmed at the in vitro level that CEACAM1-4L-overexpressing MSCs can not only significantly suppress the proliferation of T cells, but also cytokine secretion through the above examples, in order to verify whether CEACAM1-4L-overexpressing MSCs are also effective against T-cell-mediated diseases at the in vim level, in vivo experiments were performed by selecting a graft-versus-host disease (GvHD) model. GvHD is a disease that appears when donor T cells attack recipient cells, and it is important to suppress the activity of donor T cells during the onset of GvHD. To induce GvHD in a mouse model, the present inventors irradiated 9-week-old NSG mice with 2.0 Gy radiation and then intravenously injected 2.5-10′ human PBMCs. The body weight of GvHD-induced mice began to decrease from day 15, and to confirm the therapeutic effect of CEACM1-overexpressing MSCs, a vehicle, empty-MSCs, CEACM1-4L-overexpressing MSCs, and CEACM1-4S-overexpressing MSCs were injected intravenously on day 18.

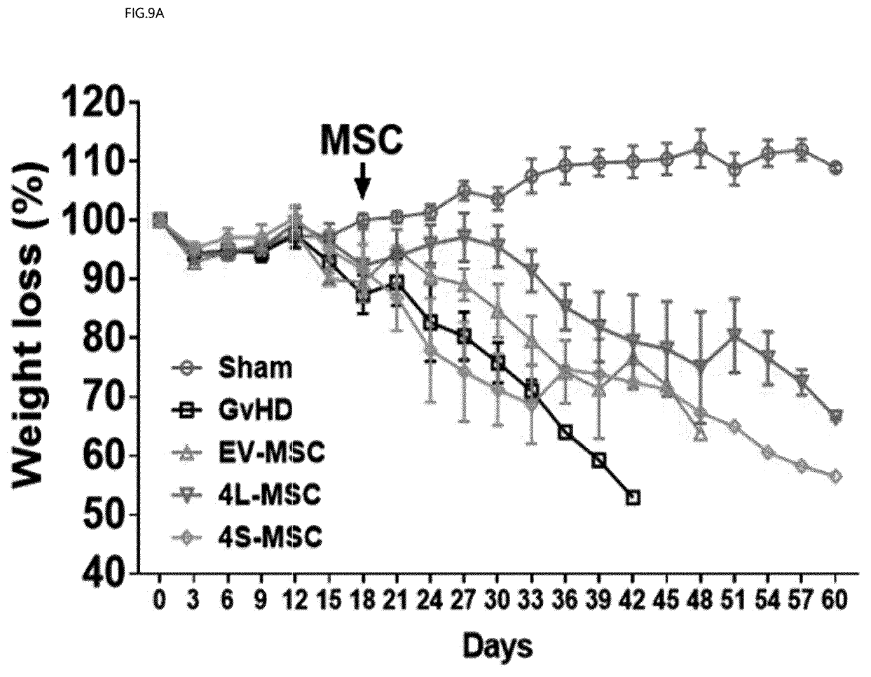

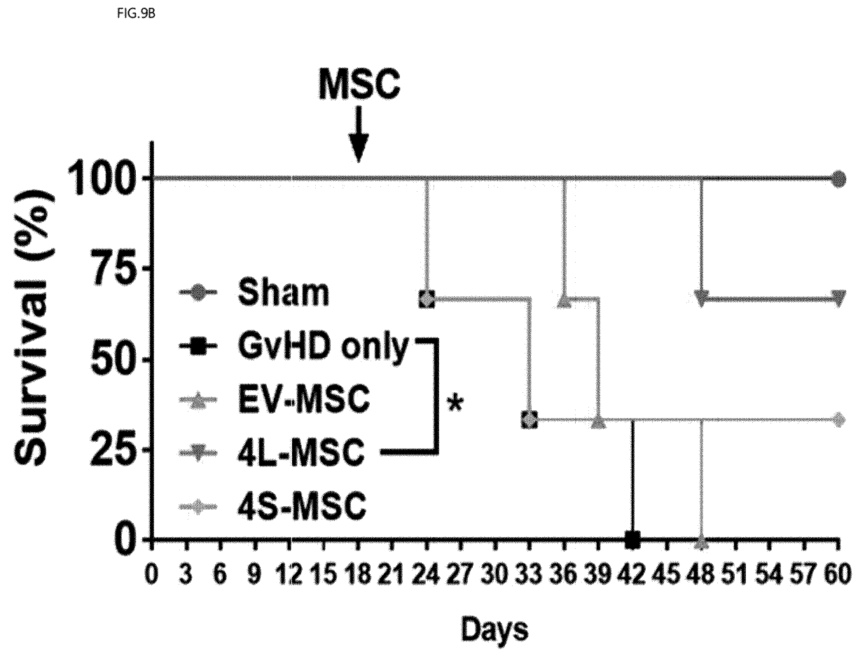

As a result of observing changes in body weight, the empty-MSC-injected group showed a tendency to gain weight again, but lost body weight at a rate similar to that of non-treated mice (GvHD group). In contrast, in the CEACAM1-4L-overexpressing MSC-injected group, the body weight increased again until day 27, and decreased from day 30, showing a more gradual decrease than the untreated mice (GvHD group). The CEACAM1-4S-overexpressing MSC-injected group lost body weight at almost the same rate as untreated mice (GvHD group), but the body weight loss decreased at a gradual rate from day 36 (FIG. 9A). In addition, the survival rate analysis also showed a significantly increased survival rate in the CEACAM1-4L overexpressing MSC-injected group compared to untreated mice (GvHD group)(FIG. 9B). These results indicate that CEACAM1-4L-overexpressing MSCs are the most effective in regulating T cell activity in vivo, similar to in vitro experiments, and that CEACAM1-4L-overexpressing MSCs increase the GvHD therapeutic effect.

Through the above examples, the present inventors confirmed that CEACAM1-overexpressing MSCs can effectively suppress the early stage of CD4 T cell proliferation and the excessive division of CD8 T cells, and that the cytokine secretion of CD4 T cells and CD8 T cells, which causes a decrease in MSC survival rate, can be suppressed. Furthermore, through specific experiments using a graft-versus-host disease animal model, it was confirmed that CEACAM1-overexpressing MSCs can actually treat T-cell-mediated inflammatory diseases. Therefore, the CEACAM1-overexpressing MSCs according to the present invention are expected to be used as an effective cell therapy agent for immunological diseases such as graft-versus-host disease, various inflammatory diseases such as asthma and fibrosis, and various immune cell-related diseases such as autoimmune diseases.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are only exemplary in all aspects and are not restrictive.

### Industrial Applicability

A stem cell and a pharmaceutical composition comprising the same as an active ingredient, according to the present invention, have the effects of reducing the degranulation and cell-killing activity of NK cells and increasing the survival rate of stem cells. Therefore, it is expected that if a stem cell in which CEACAM protein is overexpressed and an immune escape function is obtained, according to the present invention, repeated administration-induced side effects are reduced by increasing the in vivo survival rate thereof and, simultaneously, use as a cell therapeutic agent effective for various inflammatory diseases, autoimmune diseases, and the like, such as graft-versus-host disease, asthma, and fibrosis is possible.

1. A method for treating a cell damage-related disease, comprising:
administering a composition to a subject in need thereof,
wherein the composition comprises, as an active ingredient, one or more selected from the group consisting of stem cells genetically engineered to overexpress a carcinoembryonic antigen-related cell adhesion molecule (CEACAM) family protein, cells differentiated from the stem cells, and components derived from the stem cells.

2. The method of claim 1, wherein the CEACAM family protein is one or more selected from the group consisting of CEACAM1, CEACAM3, CEACAM5, and CEACAM6.

3. The method of claim 1, wherein the CEACAM family protein is CEACAM1.

4. The stem-eel method of claim 3, wherein the CEACAM1 is one or more selected from the group consisting of CEACAM1-3L, CEACAM1-3S, CEACAM1-4L and CEACAM1-4S.

5. The method of claim 1, wherein the stem cell is mesenchymal stem cells.

6. The method of claim 5, wherein the mesenchymal stem cells are one or more selected from the group consisting of bone marrow-derived mesenchymal stem cells, adipose-derived mesenchymal stem cells, umbilical cord-derived mesenchymal stem cells, umbilical cord blood-derived mesenchymal stem cells, embryonic stem cell-derived mesenchymal stem cells, and induced pluripotent stem cell-derived mesenchymal stem cells.

7. The method of claim 1, wherein the stem cell further overexpresses an immune checkpoint protein.

8. The method of claim 7, wherein the immune checkpoint protein is one or more selected from the group consisting of PD-1, PD-L1, PD-L2, CD47, CD39, CD73, CD200, HVEM, CD155, TIM3, LAG-3, CTLA-4, A2AR, B7-H3, B7-H4, HLA-E, BTLA, IDO, KIR, and VISTA.

9. The method of claim 1, wherein the stem cell is capable of evading an immune response by natural killer (NK) cells, suppresses the proliferation of T cells, or has an improved in vivo survival rate.

10. The method of claim 9, wherein the evasion of the immune response by NK cells is caused by a decrease in degranulation activity of NK cells.

11-13. (canceled)

14. The method of claim 1, wherein the cell damage-related disease is one or more selected from the group consisting of inflammatory diseases, autoimmune diseases, neurodegenerative diseases, and graft-versus-host disease.

15. The method claim 14, wherein the inflammatory disease is any one or more selected from the group consisting of atopic dermatitis, systemic lupus erythematosus, lupus, lupus pernio, lupus tuberculosis, lupus nephritis, dystrophic epidermolysis bullosa, psoriasis, rheumatic fever, rheumatoid arthritis, lumbago, fibromyalgia, myofascial diseases, undifferentiated spondyloarthropathy, undifferentiated arthropathy, arthritis, inflammatory osteolysis, reactive arthritis, osteoarthritis, scleroderma, osteoporosis, chronic inflammatory diseases caused by viral or bacterial infection, colitis, ulcerative colitis, inflammatory bowel disease, fungal infections, burns, wounds caused by surgical or dental surgery, diabetic foot ulcers, type 1 diabetes, type2 diabetes, ulcerative skin diseases, sinusitis, rhinitis, conjunctivitis, asthma, dermatitis, inflammatory collagen vascular disease, glomerulonephritis, encephalitis, inflammatory enteritis, chronic obstructive pulmonary disease, bronchiolitis obliterans, sepsis, septic shock, pulmonary fibrosis, atherosclerosis, myocarditis, endocarditis, pericarditis, cystic fibrosis, Hashimoto's thyroiditis, Graves' disease, leprosy, syphilis, Lyme disease, Borreliosis, neurological-Borreliosis, tuberculosis, sarcoidosis, macular degeneration, uveitis, irritable bowel syndrome, Crohn's disease, Sjogren's syndrome, chronic fatigue syndrome, chronic fatigue immune dysfunction syndrome, myalgic encephalomyelitis, amyotrophic lateral sclerosis, Parkinson's disease, and multiple sclerosis.

16. The method of claim 14, wherein the autoimmune disease is any one or more selected from the group consisting of autoimmune hepatitis, rheumatoid arthritis, osteoarthritis, insulin dependent diabetes mellitus, ulcerative colitis, Crohn's disease, multiple sclerosis, autoimmune myocarditis, scleroderma, myasthenia gravis, polymyositis, dermatomyositis, Hashimoto's disease, autoimmune cytopenia, Sjogren's syndrome, vasculitis syndrome, and systemic lupus erythematosus.

17. The method of claim 14, wherein the neurodegenerative disease is any one or more selected from the group consisting of Alzheimer's disease, dementia, multi-infarct dementia, frontotemporal dementia, dementia with Lewy bodies, mild cognitive impairment, corticobasal degeneration, Parkinson's disease, depression, metabolic brain diseases, multiple system atrophy, Huntington's disease, progressive supranuclear palsy, epilepsy, spinal muscular atrophy, dentatorubropallidoluysian atrophy, spinocerebellar ataxia, glaucoma, stroke, cerebral ischemia, postencephalitic parkinsonism Tourette syndrome, restless leg syndrome, attention-deficit hyperactivity disorder, Kennedy's disease, amyotrophic lateral sclerosis, multiple sclerosis, primary lateral sclerosis, and progressive bulbar palsy.

18. (canceled)

19. A method for preparing a stem cell genetically engineered to overexpress a carcinoembryonic antigen-related cell adhesion molecule (CEACAM) family protein, the method comprising:
cloning a viral vector expressing a CEACAM family protein;
preparing a vims comprising the viral vector; and
overexpressing the CEACAM family protein by infecting stem cells with the virus.

20. The method of claim 19, wherein the viral vector is one or more selected from the group consisting of a lentiviral vector, a retroviral vector, an adenoviral vector, and a paramyxovirus vector.

21-23. (canceled)

24. A stem cell genetically engineered to overexpress a carcinoembryonic antigen-related cell adhesion molecule (CEACAM) family protein to obtain an ability to evade immune responses.
